(19) United States
(12) Patent Application Publication
(10) Pub. No.: US 2024/0237899 A1
(43) Pub. Date: Jul. 18, 2024

(54) EFFICIENT TIME MULTIPLEXING IN FLUORESCENCE AND SPECTRAL IMAGING

(71) Applicant: Cilag GmbH International, Zug (CH)

(72) Inventors: Ling XU, Belmont, CA (US); Jeremiah D. Henley, Fair Oaks, CA (US)

(73) Assignee: Cilag GmbH International, Zug (CH)

(21) Appl. No.: 18/153,938

(22) Filed: Jan. 12, 2023

(51) Int. Cl. A61B 5/00; A61B 1/04; A61B 1/05; A61B 1/06

(52) U.S. Cl. CPC A61B 5/0071; A61B 1/043; A61B 1/05; A61B 1/0638

(57) ABSTRACT

Visualization systems with customizable illumination cycles to account for relative brightness disparities between different illumination sources. A system includes an image sensor comprising a pixel array and an emitter comprising a plurality of sources of electromagnetic radiation, wherein the plurality of sources comprises a first source and a second source. The emitter cycles at least a portion of the plurality of sources according to a pulse cycle comprising a plurality of pulsed emissions by the first source and a plurality of pulsed emissions by the second source. At least a portion of the plurality of pulsed emissions by the second source overlap a readout period of the image sensor when the image sensor reads out a data frame corresponding with an emission by the first source.

### TECHNICAL FIELD

This disclosure is directed to advanced visualization and digital imaging systems and methods and, more particularly but not entirely, to efficient time multiplexing of different illumination sources.

### BACKGROUND

Endoscopic surgical instruments are often preferred over traditional open surgical devices because the small incision tends to reduce post-operative recovery time and associated complications. In some instances of endoscopic visualization, it is desirable to view a space with high-definition color imaging and further with one or more advanced visualization techniques providing additional information that cannot be discerned with the human eye. However, the space constrained environment of an endoscope introduces numerous technical challenges when seeking to capture advanced visualization data in a light deficient environment.

There are numerous endoscopic visualization systems that seek to capture advanced visualization data, such as multispectral data, fluorescence data, and laser mapping data, while working within the space constrained environment of an endoscope. However, these endoscopic visualization systems do not address the inherent qualities of an image sensor that lead to a pixel array being more or less efficient at accumulating electromagnetic radiation at different wavebands across the electromagnetic spectrum.

Traditional fluorescence visualization systems implementing time multiplexed illumination may pulse white light and fluorescence excitation light for equal durations. In a traditional example implementation, a system instructs a white light source to emit white light for a defined duration and then instruct an image sensor to read out a pixel array. The system may then instruct a fluorescence light source to emit a fluorescence excitation light for the same defined duration, and then instruct the image sensor to read out the pixel array. Thus, in this traditional multiplexed visualization system, the white light source and the fluorescence light source are instructed to emit light at different times and for the same defined duration.

The traditional visualization system described above does not consider the disparity in brightness or energy output by the white light source and the fluorescence light source. The traditional visualization system further does not account for a pixel array inherently possessing varying sensitivity to different wavebands of light. In most cases, a fluorescence light source is relatively dim when compared with a white light source, and thus, the image sensor struggles to accumulate sufficient energy when exposed to the fluorescence excitation light. In the traditional visualization system, the image frames output in response to an emission by the fluorescence light source are dimmer and have lower accumulated energy levels when compared with the image frames output in response to an emission by the white light source. The traditional visualization system may attempt to correct for the energy disparity with digital post-processing of images but does not account for the energy disparity during image acquisition.

For example, commonly owned U.S. Patent Application Publication No. 2020/0404131, entitled “HYPERSPECTRAL AND FLUORESCENCE IMAGING WITH TOPOLOGY LASER SCANNING IN A LIGHT DEFICIENT ENVIRONMENT,” filed on Oct. 24, 2019, which is incorporated by reference in its entirety, describes an endoscopic visualization system for color and “specialty” imaging. In this disclosure, an emitter is configured to emit red, green, blue, and specialty emissions of light, wherein the specialty emissions may include hyperspectral, fluorescence, or laser mapping emissions. However, this disclosure does not indicate the durations of light emissions may be optimized or adjusted based on the pixel array's inherent sensitivities to different wavebands of electromagnetic radiation. Additionally, this disclosure does not indicate that multiple light sources may be emitted simultaneously to enable an image sensor to accumulate more energy from dimmer light sources.

Further for example, U.S. Pat. No. 10,958,852, entitled “IMAGING APPARATUS AND CONTROL METHOD HAVING A PLURALITY OF SYNCHRONIZED LIGHT SOURCE DEVICES,” filed on Sep. 25, 2019, which is incorporated by reference in its entirety, describes an imaging apparatus with three image sensors that receive illumination from a white light source and an infrared laser. In this disclosure, the three image sensors are dedicated to sensing green, blue, or a combination of red and infrared light. The white light source and the infrared laser may be actuated according to various patterns, including wherein the infrared laser is shown continuously and the white light source is turned on and off. However, this disclosure does not indicate wherein the white light source and the infrared laser may be synchronized such that pulses of the infrared laser overlap readout periods for color image frames, but do not overlap emissions of the white light source. Additionally, this disclosure does not indicate wherein two image sensors may sense color and infrared visualization data for stereo visualization.

Consequently, a significant need exists for a visualization system that can capture color, including high-definition color, multispectral, fluorescence, and laser mapping imaging data with consistent exposure from frame to frame despite energy disparities between white, multispectral, fluorescence, and laser mapping light sources. Specifically, there is a need to extend a duration of time an image sensor is exposed to weaker light sources without sacrificing the quality of resultant image frames.

In view of the foregoing, described herein are systems, methods, and devices for fluorescence, multispectral, and laser mapping imaging in a light deficient environment, wherein weaker light sources may be emitted simultaneously with stronger light sources.

### BRIEF DESCRIPTION OF THE DRAWINGS

Non-limiting and non-exhaustive implementations of the disclosure are described with reference to the following figures, wherein like reference numerals refer to like parts throughout the various views unless otherwise specified. Advantages of the disclosure will become better understood with regard to the following description and accompanying drawings where:

FIG. 1A is a schematic illustration of an example system for endoscopic visualization with color imaging and advanced imaging;

FIG. 1B is a schematic illustration of an example image pickup portion of a system for endoscopic visualization with color imaging and advanced imaging

FIG. 1C is a schematic illustration of an example emitter and controller of a system for endoscopic visualization with color imaging and advanced imaging;

FIG. 2A is a schematic block diagram of an example data flow for a time-sequenced visualization system;

FIG. 2B is a schematic block diagram of an example data flow for a time-sequenced visualization system;

FIG. 2C is a schematic flow chart diagram of a data flow for capturing and reading out data for a time-sequenced visualization system;

FIG. 3A is a schematic block diagram of an example system for processing data output by an image sensor with a controller in communication with an emitter and the image sensor;

FIG. 3B is a schematic block diagram of an example system for processing data output by an image sensor to generate color imaging data and advanced imaging data;

FIG. 3C is a schematic block diagram of an example system for processing data through a memory buffer to provide data frames to an image signal processor at regular intervals;

FIG. 4 is a schematic diagram of an illumination system for illuminating a light deficient environment according to a variable pulse cycle;

FIG. 5 is a schematic illustration of a synchronized operational cycle of an emitter and an image sensor;

FIG. 6 is a schematic illustration of a synchronized operational cycle of an emitter and an image sensor;

FIG. 7 is a schematic illustration of a synchronized operational cycle of an emitter and an image sensor;

FIG. 8A is a schematic illustration of an example mapping pattern comprising a grid array;

FIG. 8B is a schematic illustration of an example mapping pattern comprising a dot array;

FIG. 9 illustrates a portion of the electromagnetic spectrum divided into a plurality of different wavebands which may be pulsed by sources of electromagnetic radiation of an emitter;

FIG. 10 is a schematic diagram illustrating a timing sequence for emission and readout for generating data frames in response to pulses of electromagnetic radiation; and

FIG. 11 is a schematic block diagram of an example computing device.

### DETAILED DESCRIPTION

Disclosed herein are systems, methods, and devices for digital visualization that may be primarily suited to medical applications such as medical endoscopic imaging. An embodiment of the disclosure is an endoscopic system for color visualization and “advanced visualization” of a scene. The advanced visualization includes one or more of multispectral imaging, fluorescence imaging, or topographical mapping. Data retrieved from the advanced visualization may be processed by one or more algorithms configured to determine characteristics of the scene. The advanced visualization data may specifically be used to identify tissue structures within a scene, generate a three-dimensional topographical map of the scene, calculate dimensions of objects within the scene, identify margins and boundaries of different tissue types, and so forth.

The systems, methods, and devices described herein are specifically optimized to account for variations between “stronger” electromagnetic radiation (EMR) sources and “weaker” EMR sources. In some cases, the stronger EMR sources are considered “stronger” based on the inherent qualities of a pixel array, e.g., if a pixel array is inherently more sensitive to detecting EMR emitted by the stronger EMR source, then the stronger EMR source may be classified as “stronger” when compared with another EMR source. Conversely, if the pixel array is inherently less sensitive to detecting EMR emitted by the weaker EMR source, then the weaker EMR source may be classified as “weaker” when compared with another EMR source. Additionally, a “stronger” EMR source may have a higher amplitude, greater brightness, or higher energy output when compared with a “weaker” EMR source. The present disclosure addresses the disparity between stronger EMR sources and weaker EMR sources by adjusting a pulse cycle of an emitter to ensure a pixel array has sufficient time to accumulate a sufficient amount of EMR corresponding with each of a stronger EMR source and a weaker EMR source.

An embodiment of the disclosure is an endoscopic visualization system that includes an emitter, an image sensor, and a controller. The emitter includes a plurality of separate and independently actuatable sources of EMR that may be separately cycled on and off to illuminate a scene with pulses of EMR. The image sensor accumulates EMR and reads out data for generating a plurality of data frames. The controller synchronizes operations of the emitter and the image sensor to output a desired visualization scheme based on user input. The visualization scheme may include a selection of one or more of color imaging, multispectral imaging, fluorescence imaging, topographical mapping, or anatomical measurement.

The controller instructs the emitter and the image sensor to operate in a synchronized sequence to output a video stream that includes one or more types of visualization (i.e., color imaging, multispectral imaging, fluorescence imaging, topographical mapping, or anatomical measurement). The controller instructs the emitter to actuate one or more of the plurality of EMR sources to pulse according to a variable pulse cycle. The controller instructs the image sensor to accumulate EMR and read out data according to a variable sensor cycle that is synchronized in time with the variable pulse cycle. The synchronized sequence of the emitter and the image sensor enables the image sensor to read out data corresponding with a plurality of different visualization types. For example, the image sensor may read out a color frame in response to the emitter pulsing a white light or other visible EMR, the image sensor may readout a multispectral frame in response to the emitter pulsing a multispectral waveband of EMR, the image sensor may read out data for calculating a three-dimensional topographical map in response to the emitter pulsing EMR in a mapping pattern, and so forth.

The controller optimizes and adjusts a sensor cycle of an image sensor to output data frames for color imaging and/or advanced imaging at a sufficient rate, while ensuring the pixel array accumulates a sufficient amount of EMR for each data frame. The controller may instruct the image sensor to implement pixel binning on a per-frame basis, such that the image sensor implements pixel binning for some data frames and reads out all pixels for other data frames. In some cases, the controller instructs the image sensor to read out all pixels and thereby output a high-definition color data frame in response to the emitter pulsing white EMR. The controller may further instruct the image sensor to bin the pixel array and read out fewer pixels in response to the emitter pulsing EMR for advanced visualization, such as multispectral imaging, fluorescence imaging, or topographical mapping.

The controller may additionally optimize and adjust the variable pulse cycle in real-time based on user input, sufficient exposure of resultant data frames, and inherent properties of a corresponding pixel array. In some cases, a pixel array has varying sensitivities to different wavebands of EMR. In these cases, the pixel array is irradiated with EMR for shorter or longer durations of time depending on the type of illumination pulse to ensure the pixel array outputs data frames with consistent exposure levels. The controller adjusts the irradiation time of the pixel array and the pulsing duration of the emitter in real-time to compensate for the pixel array's varying efficiencies to different types of illumination.

The systems, methods, and devices described herein are implemented for color visualization and advanced visualization. The advanced visualization techniques described herein can be used to identify certain tissues, see through tissues in the foreground, calculate a three-dimensional topography of a scene, and calculate dimensions and distances for objects within the scene. The advanced visualization techniques described herein specifically include multispectral visualization, fluorescence visualization, and laser mapping visualization.

### Multispectral Visualization

Spectral imaging uses multiple bands across the electromagnetic spectrum. This is different from conventional cameras that only capture light across the three wavelengths based in the visible spectrum that are discernable by the human eye, including the red, green, and blue wavelengths to generate an RGB image. Spectral imaging may use any wavelength bands in the electromagnetic spectrum, including infrared wavelengths, the visible spectrum, the ultraviolet spectrum, x-ray wavelengths, or any suitable combination of various wavelength bands. Spectral imaging may overlay imaging generated based on non-visible bands (e.g., infrared) on top of imaging based on visible bands (e.g., a standard RGB image) to provide additional information that is easily discernable by a person or computer algorithm.

The multispectral imaging techniques discussed herein can be used to “see through” layers of tissue in the foreground of a scene to identify specific types of tissue and/or specific biological or chemical processes. Multispectral imaging can be used in the medical context to quantitatively track the process of a disease and to determine tissue pathology. Additionally, multispectral imaging can be used to identify critical structures such as nerve tissue, muscle tissue, cancerous cells, blood vessels, and so forth. In an embodiment, multispectral partitions of EMR are pulsed and data is gathered regarding the spectral responses of different types of tissue in response to the partitions of EMR. A datastore of spectral responses can be generated and analyzed to assess a scene and predict which tissues are present within the scene based on the sensed spectral responses.

Multispectral imaging enables numerous advantages over conventional imaging. The information obtained by multispectral imaging enables medical practitioners and/or computer-implemented programs to precisely identify certain tissues or conditions that may not be possible to identify with RGB imaging. Additionally, multispectral imaging may be used during medical procedures to provide image-guided surgery that enables a medical practitioner to, for example, view tissues located behind certain tissues or fluids, identify atypical cancerous cells in contrast with typical healthy cells, identify certain tissues or conditions, identify critical structures, and so forth. Multispectral imaging provides specialized diagnostic information about tissue physiology, morphology, and composition that cannot be generated with conventional imaging.

### Fluorescence Visualization

Fluorescence occurs when an orbital electron of a molecule, atom, or nanostructure is excited by light or other EMR, and then relaxes to its ground state by emitting a photon from the excited state. The specific frequencies of EMR that excite the orbital electron, or are emitted by the photon during relaxation, are dependent on the particular atom, molecule, or nanostructure. In most cases, the light emitted by the substance has a longer wavelength, and therefore lower energy, than the radiation that was absorbed by the substance.

Fluorescence imaging is particularly useful in biochemistry and medicine as a non-destructive means for tracking or analyzing biological molecules. The biological molecules, including certain tissues or structures, are tracked by analyzing the fluorescent emission of the biological molecules after being excited by a certain wavelength of EMR. However, relatively few cellular components are naturally fluorescent. In certain implementations, it may be desirable to visualize a certain tissue, structure, chemical process, or biological process that is not intrinsically fluorescent. In such an implementation, the body may be administered a dye or reagent that may include a molecule, protein, or quantum dot having fluorescent properties. The reagent or dye may then fluoresce after being excited by a certain wavelength of EMR. Different reagents or dyes may include different molecules, proteins, and/or quantum dots that will fluoresce at particular wavelengths of EMR. Thus, it may be necessary to excite the reagent or dye with a specialized band of EMR to achieve fluorescence and identify the desired tissue, structure, or process in the body.

The fluorescence imaging techniques described herein may be used to identify certain materials, tissues, components, or processes within a body cavity or other light deficient environment. Fluorescence imaging data may be provided to a medical practitioner or computer-implemented algorithm to enable the identification of certain structures or tissues within a body. Such fluorescence imaging data may be overlaid on black-and-white or RGB images to provide additional information and context.

The fluorescence imaging techniques described herein may be implemented in coordination with fluorescent reagents or dyes. Some reagents or dyes are known to attach to certain types of tissues and fluoresce at specific wavelengths of the electromagnetic spectrum. In an implementation, a reagent or dye is administered to a patient that is configured to fluoresce when activated by certain wavelengths of light. The visualization system disclosed herein is used to excite and fluoresce the reagent or dye. The fluorescence of the reagent or dye is detected by an image sensor to aid in the identification of tissues or structures in the body cavity. In an implementation, a patient is administered a plurality of reagents or dyes that are each configured to fluoresce at different wavelengths and/or provide an indication of different structures, tissues, chemical reactions, biological processes, and so forth. In such an implementation, the visualization system described herein emits each of the applicable wavelengths to fluoresce each of the applicable reagents or dyes. This may negate the need to perform individual imaging procedures for each of the plurality of reagents or dyes.

### Laser Mapping Visualization

Laser mapping generally includes the controlled deflection of laser beams. Laser mapping can be implemented to generate one or more of a three-dimensional topographical map of a scene, calculate distances between objects within the scene, calculate dimensions of objects within the scene, track the relative locations of tools within the scene, and so forth.

Laser mapping combines controlled steering of laser beams with a laser rangefinder. By taking a distance measurement at every direction, the laser rangefinder can rapidly capture the surface shape of objects, tools, and landscapes. Construction of a full three-dimensional topography may include combining multiple surface models that are obtained from different viewing angles. Various measurement systems and methods exist in the art for applications in archaeology, geography, atmospheric physics, autonomous vehicles, and others. One such system includes light detection and ranging (LIDAR), which is a three-dimensional mapping system. LIDAR has been applied in navigation systems such as airplanes or satellites to determine position and orientation of a sensor in combination with other systems and sensors. LIDAR uses active sensors to illuminate an object and detect energy that is reflected off the object and back to a sensor.

As discussed herein, the term “laser mapping” includes laser tracking. Laser tracking, or the use of lasers for tool tracking, measures objects by determining the positions of optical targets held against those objects. Laser trackers can be accurate to the order of 0.025 mm over a distance of several meters. The visualization system described herein pulses EMR for use in conjunction with a laser tracking system such that the position of tools within a scene can be tracked and measured.

The endoscopic visualization system described herein implements laser mapping imaging to determine precise measurements and topographical outlines of a scene. In one implementation, mapping data is used to determine precise measurements between, for example, structures or organs in a body cavity, devices, or tools in the body cavity, and/or critical structures in the body cavity. As discussed herein, the term “mapping” encompasses technologies referred to as laser mapping, laser scanning, topographical scanning, three-dimensional scanning, laser tracking, tool tracking, and others. A mapping data frame as discussed herein includes data for calculating one or more of a topographical map of a scene, dimensions of objects or structures within a scene, distances between objects or structures within the scene, relative locations of tools or other objects within the scene, and so forth.

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

Before the structure, systems, and methods are disclosed and described, it is to be understood that this disclosure is not limited to the particular structures, configurations, process steps, and materials disclosed herein as such structures, configurations, process steps, and materials may vary somewhat. It is also to be understood that the terminology employed herein is used for the purpose of describing particular embodiments only and is not intended to be limiting since the scope of the disclosure will be limited only by the appended claims and equivalents thereof.

In describing and claiming the subject matter of the disclosure, the following terminology will be used in accordance with the definitions set out below.

It must be noted that, as used in this specification and the appended claims, the singular forms “a,” “an,” and “the” include plural referents unless the context clearly dictates otherwise.

As used herein, the terms “comprising,” “including,” “containing,” “characterized by,” and grammatical equivalents thereof are inclusive or open-ended terms that do not exclude additional, unrecited elements or method steps.

As used herein, the phrase “consisting of” and grammatical equivalents thereof exclude any element or step not specified in the claim.

As used herein, the phrase “consisting essentially of” and grammatical equivalents thereof limit the scope of a claim to the specified materials or steps and those that do not materially affect the basic and novel characteristic or characteristics of the claimed disclosure.

As used herein, the term “proximal” shall refer broadly to the concept of a portion nearest an origin.

As used herein, the term “distal” shall generally refer to the opposite of proximal, and thus to the concept of a portion farther from an origin, or a farthest portion, depending upon the context.

As used herein, color sensors are sensors known to have a color filter array (CFA) thereon to filter the incoming EMR into its separate components. In the visual range of the electromagnetic spectrum, such a CFA may be built on a Bayer pattern or modification thereon to separate green, red, and blue spectrum components of visible EMR.

As used herein, a monochromatic sensor refers to an unfiltered imaging sensor comprising color-agnostic pixels.

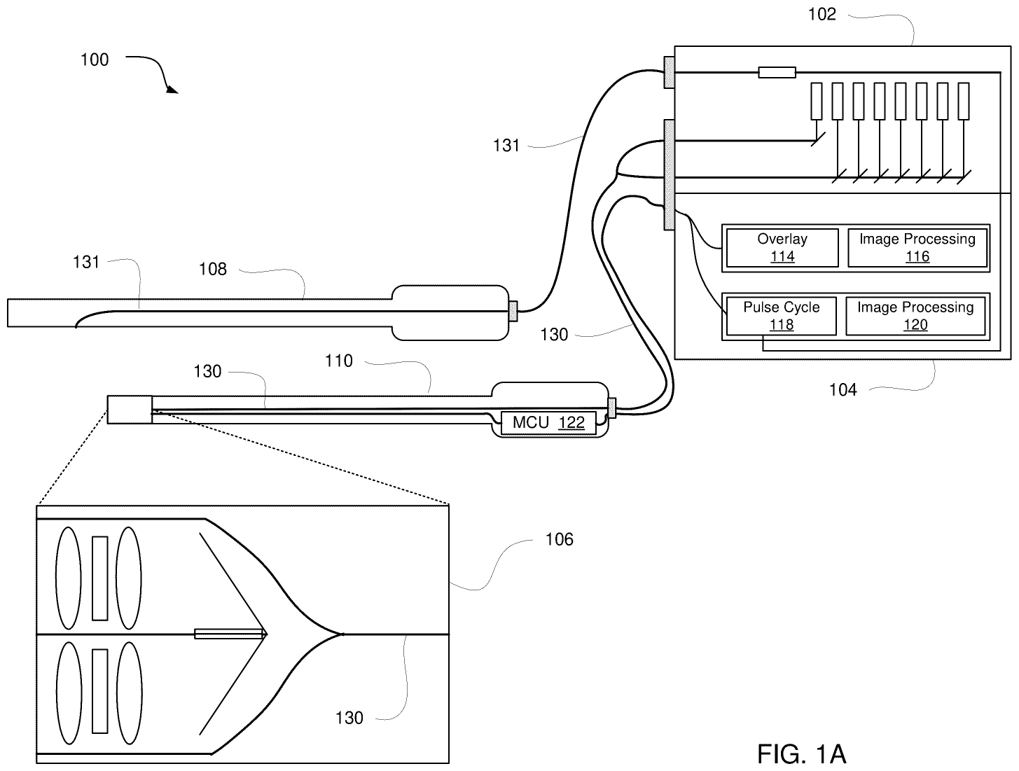

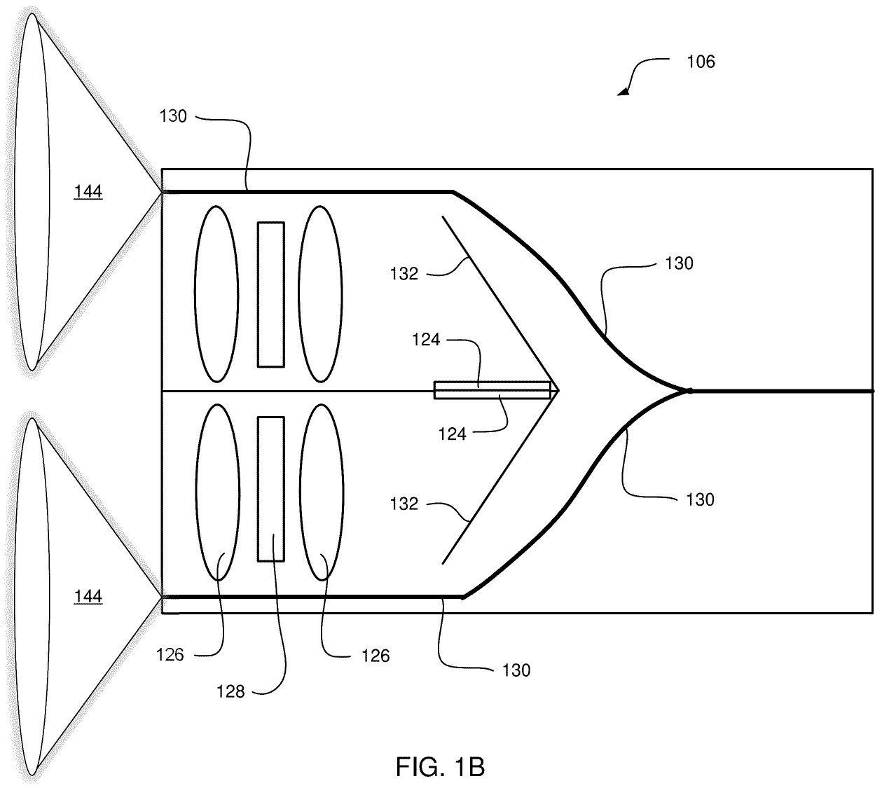

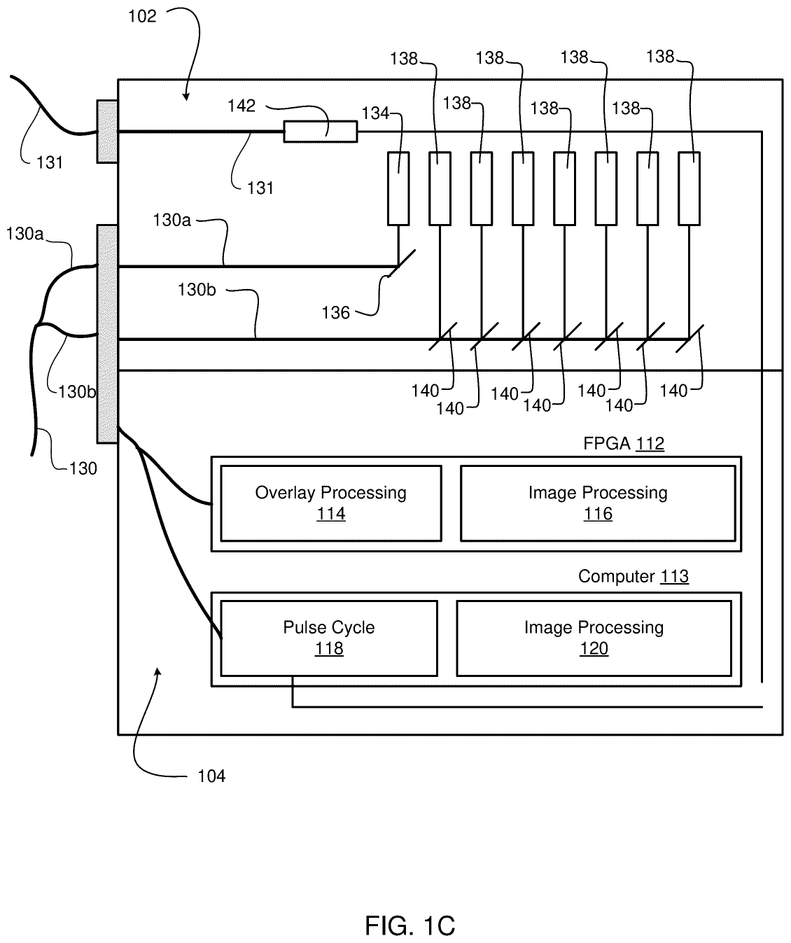

Referring now to the figures, FIGS. 1A-1C illustrate schematic diagrams of a system 100 for endoscopic visualization. The system 100 includes an emitter 102, a controller 104, and an optical visualization system 106. The system 100 includes one or more tools 108, which may include endoscopic tools such as forceps, brushes, scissors, cutters, burs, staplers, ligation devices, tissue staplers, suturing systems, and so forth. The system 100 includes one or more endoscopes 110 such as arthroscopes, bronchoscopes, colonoscopes, colposcopes, cystoscopes, esophagoscope, gastroscopes, laparoscopes, laryngoscopes, neuroendoscopes, proctoscopes, sigmoidoscopes, thoracoscopes, and so forth.

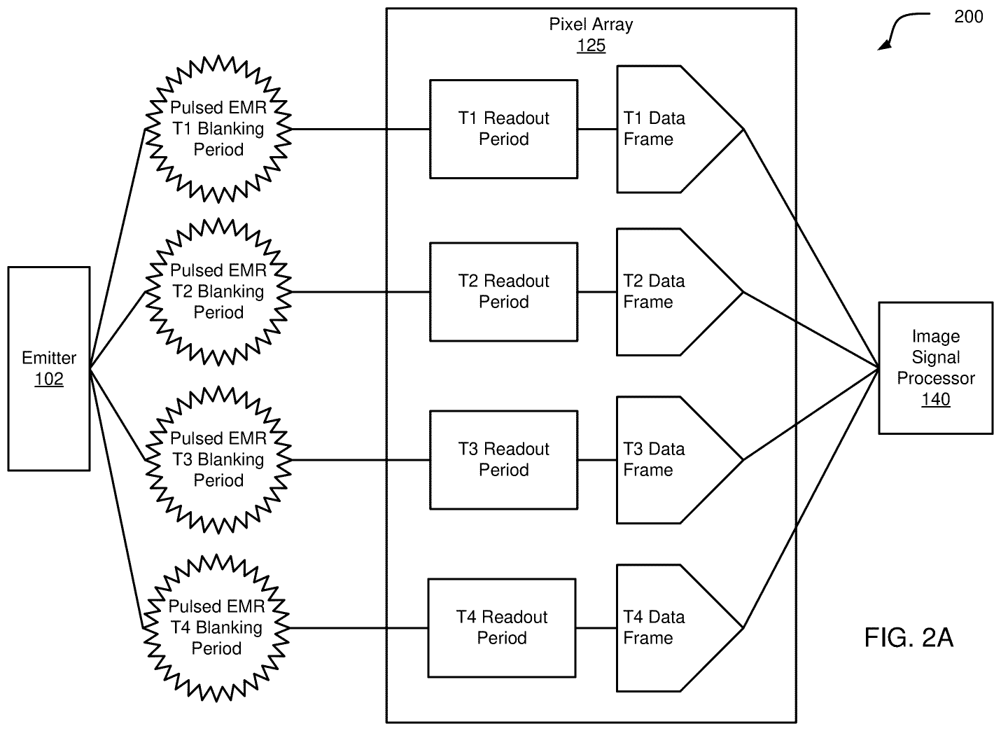

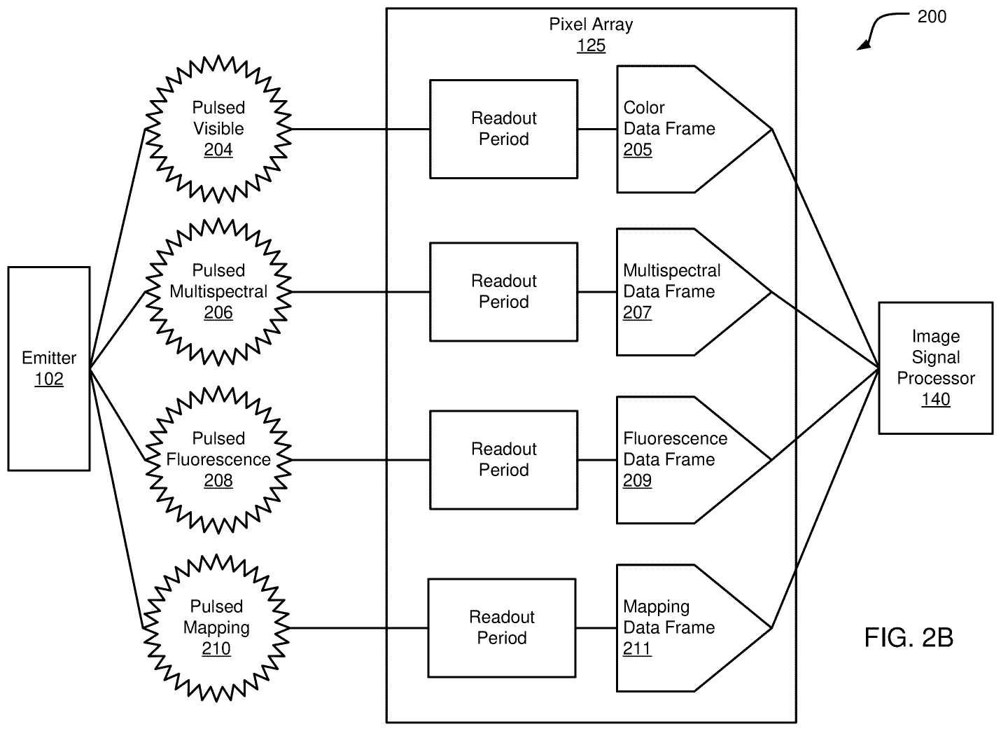

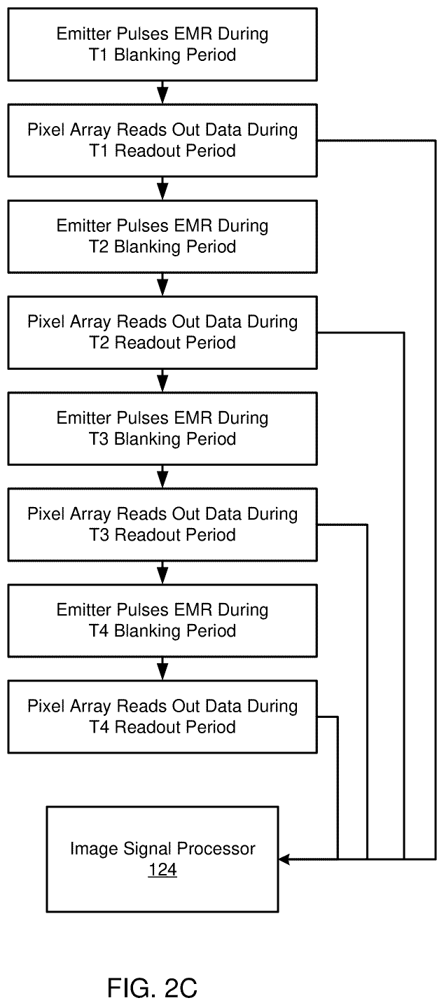

The optical visualization system 106 may be disposed at a distal end of a lumen of an endoscope 110. Alternatively, one or more components of the optical visualization system 106 may be disposed at a proximal end of the lumen of the endoscope 110 or in another region of the endoscope 110. The optical visualization system 106 may include one or more image sensors 124 that each include a pixel array (see pixel array 125 first illustrated in FIG. 2A). The optical visualization system 106 may include one or more lenses 126 and filters 128 and may further include one or more prisms 132 for reflecting EMR on to the pixel array 125 of the one or more image sensors 124. The system 100 may include a waveguide 130 configured to transmit EMR from the emitter 102 to a distal end of the endoscope 110 to illuminate a light deficient environment for visualization. The system 100 may further include a waveguide 131 configured to transmit EMR from the emitter 102 to a termination point on the tool 108, which may specifically be actuated for laser mapping imaging and tool tracking as described herein.

The optical visualization system 106 may specifically include two lenses 126 dedicated to each image sensor 124 to focus EMR on to a rotated image sensor 124 and enable a depth view. The filter 128 may include a notch filter configured to block unwanted reflected EMR. In a particular use-case, the unwanted reflected EMR may include a fluorescence excitation wavelength that was pulsed by the emitter 102, wherein the system 100 wishes to only detect a fluorescence relaxation wavelength emitted by a fluorescent reagent or tissue.

The image sensor 124 includes one or more image sensors, and the example implementation illustrated in FIGS. 1A-1B illustrates an optical visualization system 106 comprising two image sensors 124. The image sensor 124 may include a CMOS image sensor and may specifically include a high-resolution image sensor configured to read out data according to a rolling readout scheme. The image sensors 124 may include a plurality of different image sensors that are tuned to collect different wavebands of EMR with varying efficiencies. In an implementations, the image sensors 124 include separate image sensors that are optimized for color imaging, fluorescence imaging, multispectral imaging, and/or topographical mapping.

The emitter 102 includes one or more EMR sources, which may include, for example, lasers, laser bundles, light emitting diodes (LEDs), electric discharge sources, incandescence sources, electroluminescence sources, and so forth. In some implementations, the emitter 102 includes at least one white EMR source 134 (may be referred to herein as a white light source). The emitter 102 may additionally include one or more EMR sources 138 that are tuned to emit a certain waveband of EMR. The EMR sources 138 may specifically be tuned to emit a waveband of EMR that is selected for multispectral or fluorescence visualization. The emitter 102 may additionally include one or more mapping sources 142 that are configured to emit EMR in a mapping pattern such as a grid array or dot array selected for capturing data for topographical mapping or anatomical measurement.

The one or more white EMR sources 134 emit EMR into a dichroic mirror 136 that feeds the white EMR into a waveguide 130. The white EMR source 134 may specifically feed into a first waveguide 130a dedicated to white EMR. The EMR sources 138 emit EMR into independent dichroic mirrors 140 that each feed EMR into the waveguide 130 and may specifically feed into a second waveguide 130b. The first waveguide 130a and the second waveguide 130b later merge into a waveguide 130 that transmits EMR to a distal end of the endoscope 110 to illuminate a scene with an emission of EMR 144.

The one or more EMR sources 138 that are tuned to emit a waveband of EMR may specifically be tuned to emit EMR that is selected for multispectral or fluorescence visualization. In some cases, the EMR sources 138 are finely tuned to emit a central wavelength of EMR with a tolerance threshold not exceeding ±5 nm, ±4 nm, ±3 nm, ±2 nm, or ±1 nm. The EMR sources 138 may include lasers or laser bundles that are separately cycled on and off by the emitter 102 to pulse the emission of EMR 144 and illuminate a scene with a finely tuned waveband of EMR.

The one or more mapping sources 142 are configured to pulse EMR in a mapping pattern, which may include a dot array, grid array, vertical hashing, horizontal hashing, pin grid array, and so forth. The mapping pattern is selected for laser mapping imaging to determine one or more of a three-dimensional topographical map of a scene, a distance between two or more objects within a scene, a dimension of an object within a scene, a location of a tool 108 within the scene, and so forth. The EMR pulsed by the mapping source 142 is diffracted to spread the energy waves according to the desired mapping pattern. The mapping source 142 may specifically include a device that splits the EMR beam with quantum-dot-array diffraction grafting. The mapping source 142 may be configured to emit low mode laser light.

The controller 104 (may be referred to herein as a camera control unit or CCU) may include a field programmable gate array (FGPA) 112 and a computer 113. The FGPA 112 may be configured to perform overlay processing 114 and image processing 116. The computer 113 may be configured to generate a pulse cycle 118 for the emitter 102 and to perform further image processing 120. The FGPA 112 receives data from the image sensor 124 and may combine data from two or more data frames by way of overlay processing 114 to output an overlay image frame. The computer 113 may provide data to the emitter 102 and the image sensor 124. Specifically, the computer 113 may calculate and adjust a variable pulse cycle to be emitted by the emitter 102 in real-time based on user input. Additionally, the computer 113 may receive data frames from the image sensor 124 and perform further image processing 120 on those data frames.

The controller 104 may be in communication with a network, such as the Internet, and automatically upload data to the network for remote storage. The MCU 122 and image sensors 124 maybe exchanged and updated and continue to communicate with an established controller 104. In some cases, the controller 104 is “out of date” with respect to the MCU 122 but will still successfully communicate with the MCU 122. This may increase the data security for a hospital or other healthcare facility because the existing controller 104 may be configured to undergo extensive security protocols to protect patient data.

The controller 104 may reprogram the image sensor 124 for each data frame to set a required blanking period duration and/or readout period duration for a subsequent frame period. One frame period includes a blanking period and a readout period. Generally speaking, the pixel array 125 accumulates EMR during the blanking period and reads out pixel data during the readout period. It will be understood that a blanking period corresponds to a time between a readout of a last row of active pixels in the pixel array of the image sensor and a beginning of a next subsequent readout of active pixels in the pixel array. Additionally, the readout period corresponds to a duration of time when active pixels in the pixel array are being read. Further, the controller 104 may write correct registers to the image sensor 124 to adjust the duration of one or more of the blanking period or the readout period for each frame period on a frame-by-frame basis within the sensor cycle as needed.

The endoscope 110 includes a microcontroller unit (MCU) 122 disposed therein. The MCU 122 may specifically be disposed within a handpiece portion of the endoscope 110 and communicate with electronic circuitry (such as the image sensor 124) disposed within a distal end of a lumen of the endoscope 110. The MCU 122 receives instructions from the controller 104, including an indication of the pulse cycle 118 provided to the emitter 102 and the corresponding sensor cycle timing for the image sensor 124. The MCU 122 executes a common Application Program Interface (API). The controller 104 communicates with the MCU 122, and the MCU 122 executes a translation function that translates instructions received from the controller 104 into the correct format for each type of image sensor 124. In some cases, the system 100 may include multiple different image sensors that each operate according to a different “language” or formatting, and the MCU 122 is configured to translate instructions from the controller 104 into each of the appropriate data formatting languages. The common API on the MCU 122 passes information by the scene, including, for example parameters pertaining to gain, exposure, white balance, setpoint, and so forth. The MCU 122 runs a feedback algorithm to the controller 104 for any number of parameters depending on the type of visualization.

The MCU 122 stores operational data and images captured by the image sensors 124. In some cases, the MCU 122 does not need to continuously push data up the data chain to the controller 104. The data may be set once on the microcontroller 122, and then only critical information may be pushed through a feedback loop to the controller 104. The MCU 122 may be set up in multiple modes, including a primary mode (may be referred to as a “master” mode when referring to a master/slave communication protocol). The MCU 122 ensures that all downstream components (i.e., distal components including the image sensors 124, which may be referred to as “slaves” in the master/slave communication protocol) are apprised of the configurations for upcoming data frames. The upcoming configurations may include, for example, gain, exposure duration, readout duration, pixel binning configuration, and so forth.

The MCU 122 includes internal logic for executing triggers to coordinate different devices, including, for example multiple image sensors 124. The MCU 122 provides instructions for upcoming frames and executes triggers to ensure that each image sensor 124 begins to capture data the same time. In some cases, the image sensors 124 may automatically advance to a subsequent data frame without receiving a unique trigger from the MCU 122.

In some cases, the endoscope 110 includes two or more image sensors 124 that detect EMR and output data frames simultaneously. The simultaneous data frames may be used to output a three-dimensional image and/or output imagery with increased definition and dynamic range. The pixel array of the image sensor 124 may include active pixels and optical black (“OB”) or optically blind pixels. The optical black pixels may be read during a blanking period of the pixel array when the pixel array is “reset” or calibrated. After the optical black pixels have been read, the active pixels are read during a readout period of the pixel array. The active pixels accumulate EMR that is pulsed by the emitter 102 during the blanking period of the image sensor 124. The pixel array 125 may include monochromatic or “color agnostic” pixels that do not comprise any filter for selectively receiving certain wavebands of EMR. The pixel array may include a color filter array (CFA), such as a Bayer pattern CFA, that selectively allows certain wavebands of EMR to pass through the filters and be accumulated by the pixel array.

The image sensor 124 is instructed by a combination of the MCU 122 and the controller 104 working in a coordinated effort. Ultimately, the MCU 122 provides the image sensor 124 with instructions on how to capture the upcoming data frame. These instructions include, for example, an indication of the gain, exposure, white balance, exposure duration, readout duration, pixel binning configuration, and so forth for the upcoming data frame. When the image sensor 124 is reading out data for a current data frame, the MCU 122 is rewriting the correct registers for the next data frame. The MCU 122 and the image sensor 124 operate in a back-and-forth data flow, wherein the image sensor 124 provides data to the MCU 122 and the MCU 122 rewrites correct registers to the image sensor 124 for each upcoming data frame. The MCU 122 and the image sensor 124 may operate according to a “ping pong buffer” in some configurations.

The image sensor 124, MCU 122, and controller 104 engage in a feedback loop to continuously adjust and optimize configurations for upcoming data frames based on output data. The MCU 122 continually rewrites correct registers to the image sensor 124 depending on the type of upcoming data frame (i.e., color data frame, multispectral data frame, fluorescence data frame, topographical mapping data frame, and so forth), configurations for previously output data frames, and user input. In an example implementation, the image sensor 124 outputs a multispectral data frame in response to the emitter 102 pulsing a multispectral waveband of EMR. The MCU 122 and/or controller 104 determines that the multispectral data frame is underexposed and cannot successfully be analyzed by a corresponding machine learning algorithm. The MCU 122 and/or controller 104 than adjusts configurations for upcoming multispectral data frames to ensure that future multispectral data frames are properly exposed. The MCU 122 and/or controller 104 may indicate that the gain, exposure duration, pixel binning configuration, etc. must be adjusted for future multispectral data frames to ensure proper exposure. All image sensor 124 configurations may be adjusted in real-time based on previously output data processed through the feedback loop, and further based on user input.

The waveguides 130, 131 include one or more optical fibers. The optical fibers may be made of a low-cost material, such as plastic to allow for disposal of one or more of the waveguides 130, 131. In some implementations, one or more of the waveguides 130, 131 include a single glass fiber having a diameter of 500 microns. In some implementations, one or more of the waveguides 130, 131 include a plurality of glass fibers.

FIGS. 2A and 2B each illustrate a schematic diagram of a data flow 200 for time-sequenced visualization of a light deficient environment. The data flow 200 illustrated in FIGS. 2A-2B may be implemented by the system 100 for endoscopic visualization illustrated in FIGS. 1A-1C. FIG. 2A illustrates a generic implementation that may be applied to any type of illumination or wavelengths of EMR. FIG. 2B illustrates an example implementation wherein the emitter 102 actuates visible, multispectral, fluorescence, and mapping EMR sources.

The data flow 200 includes an emitter 102, a pixel array 125 of an image sensor 124 (not shown), and an image signal processor 140. The image signal processor 140 may include one or more of the image processing 116, 120 modules illustrated in FIGS. 1A and 1C. The emitter 102 includes a plurality of separate and independently actuatable EMR sources (see, e.g., 134, 138 illustrated in FIGS. 1A and 1C). Each of the EMR sources can be cycled on and off to emit a pulse of EMR with a defined duration and magnitude. The pixel array 125 of the image sensor 124 may include a color filter array (CFA) or an unfiltered array comprising color-agnostic pixels. The emitter 102 and the pixel array 125 are each in communication with a controller 104 (not shown in FIGS. 2A-2B) that instructs the emitter 102 and the pixel array 125 to synchronize operations to generate a plurality of data frames according to a desired visualization scheme.

The controller 104 instructs the emitter 102 to cycle the plurality of EMR sources according to a variable pulse cycle. The controller 104 calculates the variable pulse cycle based at least in part upon a user input indicating the desired visualization scheme. For example, the desired visualization scheme may indicate the user wishes to view a scene with only color imaging. In this case, the variable pulse cycle may include only pulses of white EMR. In an alternative example, the desired visualization scheme may indicate the user wishes to be notified when nerve tissue can be identified in the scene and/or when a tool within the scene is within a threshold distance from the nerve tissue. In this example, the variable pulse cycle may include pulses of white EMR and may further include pulses of one or more multispectral wavebands of EMR that elicit a spectral response from the nerve tissue and/or “see through” non-nerve tissues by penetrating those non-nerve tissues. Additionally, the variable pulse cycle may include pulses of EMR in a mapping pattern configured for laser mapping imaging to determine when the tool is within the threshold distance from the nerve tissue. The controller 104 may reconfigure the variable pulse cycle in real-time in response to receiving a revised desired visualization scheme from the user.

FIGS. 2A and 2B each illustrate a schematic diagram of a data flow 200 for time-sequenced visualization of a light deficient environment. The data flow 200 illustrated in FIGS. 2A-2B may be implemented by the system 100 for endoscopic visualization illustrated in FIGS. 1A-1C. FIG. 2A illustrates a generic implementation that may be applied to any type of illumination or wavelengths of EMR. FIG. 2B illustrates an example implementation wherein the emitter 102 actuates visible, multispectral, fluorescence, and mapping EMR sources.

The data flow 200 includes an emitter 102, a pixel array 125 of an image sensor 124 (not shown), and an image signal processor 140. The image signal processor 140 may include one or more of the image processing 116, 120 modules illustrated in FIGS. 1A and 1C. The emitter 102 includes a plurality of separate and independently actuatable EMR sources (see, e.g., 134, 138 illustrated in FIGS. 1A and 1C). Each of the EMR sources can be cycled on and off to emit a pulse of EMR with a defined duration and magnitude. The pixel array 125 of the image sensor 124 may include a color filter array (CFA) or an unfiltered array comprising color-agnostic pixels. The emitter 102 and the pixel array 125 are each in communication with a controller 104 (not shown in FIGS. 2A-2B) that instructs the emitter 102 and the pixel array 125 to synchronize operations to generate a plurality of data frames according to a desired visualization scheme.

The controller 104 instructs the emitter 102 to cycle the plurality of EMR sources according to a variable pulse cycle. The controller 104 calculates the variable pulse cycle based at least in part upon a user input indicating the desired visualization scheme. For example, the desired visualization scheme may indicate the user wishes to view a scene with only color imaging. In this case, the variable pulse cycle may include only pulses of broad spectrum visible EMR. In an alternative example, the desired visualization scheme may indicate the user wishes to be notified when nerve tissue can be identified in the scene and/or when a tool within the scene is within a threshold distance from the nerve tissue. In this example, the variable pulse cycle may include pulses of white EMR and may further include pulses of one or more multispectral wavebands of EMR that elicit a spectral response from the nerve tissue and/or “see through” non-nerve tissues by penetrating those non-nerve tissues. Additionally, the variable pulse cycle may include pulses of EMR in a mapping pattern configured for laser mapping imaging to determine when the tool is within the threshold distance from the nerve tissue. The controller 104 may reconfigure the variable pulse cycle in real-time in response to receiving a revised desired visualization scheme from the user.

FIG. 2A illustrates wherein the emitter cycles one or more EMR sources on and off to emit a pulse of EMR during each of a plurality of separate blanking periods of the pixel array 125. Specifically, the emitter 102 emits pulsed EMR during each of a T1 blanking period, T2 blanking period, T3 blanking period, and T4 blanking period of the pixel array 125. The pixel array 125 accumulates EMR during its blanking periods and reads out data during its readout periods.

Specifically, the pixel array 125 accumulates EMR during the T1 blanking period and reads out the T1 data frame during the T1 readout period, which follows the T1 blanking period. Similarly, the pixel array 125 accumulates EMR during the T2 blanking period and reads out the T2 data frame during the T2 readout period, which follows the T2 blanking period. The pixel array 125 accumulates EMR during the T3 blanking period and reads out the T3 data frame during the T3 readout period, which follows the T3 blanking period. The pixel array 125 accumulates EMR during the T4 blanking period and reads out the T4 data frame during the T4 readout period, which follows the T4 blanking period. Each of the T1 data frame, the T2 data frame, the T3 data frame, and the T4 data frame is provided to the image signal processor 140.

The contents of each of the T1-T4 data frames is dependent on the type of EMR that was pulsed by the emitter 102 during the preceding blanking period. For example, if the emitter 102 pulses white light during the preceding blanking period, then the resultant data frame may include a color data frame (if the pixel array 125 includes a color filter array for outputting red, green, and blue image data). Further for example, if the emitter 102 pulses a multispectral waveband of EMR during the preceding blanking period, then the resultant data frame is a multispectral data frame comprising information for identifying a spectral response by one or more objects within the scene and/or information for “seeing through” one or more structures within the scene. Further for example, if the emitter 102 pulses a fluorescence excitation waveband of EMR during the preceding blanking period, then the resultant data frame is a fluorescence data frame comprising information for identifying a fluorescent reagent or autofluorescence response by a tissue within the scene. Further for example, if the emitter 102 pulses EMR in a mapping pattern during the preceding blanking period, then the resultant data frame is a mapping data frame comprising information for calculating one or more of a three-dimensional topographical map of the scene, a dimension of one or more objects within the scene, a distance between two or more objects within the scene, and so forth.

Some “machine vision” or “computer vision” data frames, including multispectral data frames, fluorescence data frames, and mapping data frames may be provided to a corresponding algorithm or neural network configured to evaluate the information therein. A multispectral algorithm may be configured to identify one or more tissue structures within a scene based on how those tissue structures respond to one or more different wavebands of EMR selected for multispectral imaging. A fluorescence algorithm may be configured to identify a location of a fluorescent reagent or auto-fluorescing tissue structure within a scene. A mapping algorithm may be configured to calculate one or more of a three-dimensional topographical map of a scene, a depth map, a dimension of one or more objects within the scene, and/or a distance between two or more objects within the scene based on the mapping data frame.

FIG. 2B illustrates an example wherein the emitter 102 cycles separate visible, multispectral, fluorescence, and mapping EMR sources to emit pulsed visible 204, pulsed multispectral 206, pulsed fluorescence 208, and pulsed EMR in a mapping pattern 210. It should be appreciated that FIG. 2B is illustrative only, and that the emissions 204, 206, 208, 210 may be emitted in any order, may be emitted during a single visualization session as shown in FIG. 2B, and may be emitted during separate visualization sessions.

The pixel array 125 reads out a color data frame 205 in response to the emitter 102 pulsing the pulsed visible 204 EMR. The pulsed visible 204 EMR may specifically include a pulse of white light. The pixel array 125 reads out a multispectral data frame 207 in response to the emitter 102 pulsing the multispectral 206 waveband of EMR. The pulsed multispectral 206 waveband of EMR may specifically include one or more of EMR within a waveband from about 513-545 nanometers (nm), 565-585 nm, 770-790 nm, and/or 900-1000 nm. It will be appreciated that the pulsed multispectral 206 waveband of EMR may include various other wavebands used to elicit a spectral response. The pixel array 125 reads out a fluorescence data frame 209 in response to the emitter 102 pulsing the fluorescence 208 waveband of EMR. The pulsed fluorescence 208 waveband of EMR may specifically include one or more of EMR within a waveband from about 770-795 nm and/or 790-815 nm. The pixel array 125 reads out a mapping data frame 211 in response to the emitter 102 pulsing EMR in a mapping pattern 210. The pulsed mapping pattern 210 may include one or more of vertical hashing, horizontal hashing, a pin grid array, a dot array, a raster grid of discrete points, and so forth. Each of the color data frame 205, the multispectral data frame 207, the fluorescence data frame 209, and the mapping data frame 211 is provided to the image signal processor 140.

In an implementation, the emitter 102 separately pulses red, green, and blue visible EMR. In this implementation, the pixel array 125 may include a monochromatic (color agnostic) array of pixels. The pixel array 125 may separately read out a red data frame, a green data frame, and a blue data frame in response to the separate pulses of red, green, and blue visible EMR.

In an implementation, the emitter 102 separately pulses wavebands of visible EMR that are selected for capturing luminance (“Y”) imaging data, red chrominance (“Cr”) imaging data, and blue chrominance (“Cb”) imaging data. In this implementation, the pixel array 125 may separately read out a luminance data frame (comprising only luminance imaging information), a red chrominance data frame, and a blue chrominance data frame.

FIG. 2C illustrates a schematic flow chart diagram of a process flow for synchronizing operations of the emitter 102 and the pixel array 125. The process flow corresponds with the schematic diagram illustrated in FIG. 2A. The process flow includes the controller 104 instructing the emitter 102 to pulse EMR during a T1 blanking period of the pixel array 125 and then instructing the pixel array 125 to read out data during a T1 readout period following the T1 blanking period. Similarly, the controller 104 instructs the emitter to pulse EMR during each of the T2 blanking period, the T3 blanking period, and the T4 blanking period. The controller 104 instructs the emitter to read out data during each of the T2 readout period, the T3 readout period, and the T4 readout period that follow the corresponding blanking periods. Each of the output data frames are provided to the image signal processor 140.

The emitter 102 pulses according to a variable pulse cycle that includes one or more types of EMR. The variable pulse cycle may include visible EMR, which may include a white light emission, red light emission, green light emission, blue light emission, or some other waveband of visible EMR. The white light emission may be pulsed with a white light emitting diode (LED) or other light source and may alternatively be pulsed with a combination of red, green, and blue light sources pulsing in concert. The variable pulse cycle may include one or more wavebands of EMR that are selected for multispectral imaging or fluorescence imaging. The variable pulse cycle may include one or more emissions of EMR in a mapping pattern selected for three-dimensional topographical mapping or calculating dimensions within a scene. In some cases, several types of EMR are represented in the variable pulse cycle with different regularity than other types of EMR. This may be implemented to emphasize and de-emphasize aspects of the recorded scene as desired by the user.

The controller 104 adjusts the variable pulse cycle in real-time based on the visualization objectives. The system enables a user to input one or more visualization objectives and to change those objectives while using the system. For example, the visualization objective may indicate the user wishes to view only color imaging data, and in this case, the variable pulse cycle may include pulsed or constant emissions of white light (or other visible EMR). The visualization objective may indicate the user wishes to be notified when a scene includes one or more types of tissue or conditions that may be identified using one or more of color imaging, multispectral imaging, or fluorescence imaging. The visualization objective may indicate that a patient has been administered a certain fluorescent reagent or dye, and that fluorescence imaging should continue while the reagent or dye remains active. The visualization objective may indicate the user wishes to view a three-dimensional topographical map of a scene, receive information regarding distances or dimensions within the scene, receive an alert when a tool comes within critical distance from a certain tissue structure, and so forth.

The variable pulse cycle may include one or more finely tuned partitions of the electromagnetic spectrum that are selected to elicit a fluorescence response from a reagent, dye, or auto-fluorescing tissue. The fluorescence excitation wavebands of EMR include one or more of the following: 400±50 nm, 450±50 nm, 500±50 nm, 550±50 nm, 600±50 nm, 650±50 nm, 700±50 nm, 710±50 nm, 720±50 nm, 730±50 nm, 740±50 nm, 750±50 nm, 760±50 nm, 770±50 nm, 780±50 nm, 790±50 nm, 800±50 nm, 810±50 nm, 820±50 nm, 830±50 nm, 840±50 nm, 850±50 nm, 860±50 nm, 870±50 nm, 880±50 nm, 890±50 nm, or 900±50 nm. The aforementioned wavebands may be finely tuned such that the emitter pulses the central wavelength with a tolerance threshold of ±100 nm, ±90 nm, ±80 nm, ±70 nm, ±60 nm, ±50 nm, ±40 nm, ±30 nm, ±20 nm, ±10 nm, ±8 nm, ±6 nm, ±5 nm, ±4 nm, ±3 nm, ±2 nm, ±1 nm, and so forth. In some cases, the emitter includes a plurality of laser bundles that are each configured to pulse a particular wavelength of EMR with a tolerance threshold not greater than ±5 nm, ±4 nm, ±3 nm, or ±2 nm.

The variable pulse cycle may include one or more wavebands of EMR that are tuned for multispectral imaging. These wavebands of EMR are selected to elicit a spectral response from a certain tissue or penetrate through a certain tissue (such that substances disposed behind that tissue may be visualized). The multispectral wavebands of EMR include one or more of the following: 100±50 nm, 150±50 nm, 200±50 nm, 250±50 nm, 300±50 nm, 350±50 nm, 400±50 nm, 450±50 nm, 400±50 nm, 410±50 nm, 420±50 nm, 430±50 nm, 440±50 nm, 450±50 nm, 460±50 nm, 470±50 nm, 480±50 nm, 490±50 nm, 500±50 nm, 510±50 nm, 520±50 nm, 530±50 nm, 540±50 nm, 550±50 nm, 560±50 nm, 570±50 nm, 580±50 nm, 590±50 nm, 600±50 nm, 610±50 nm, 620±50 nm, 630±50 nm, 640±50 nm, 650±50 nm, 660±50 nm, 670±50 nm, 680±50 nm, 690±50 nm, 700±50 nm, 710±50 nm, 720±50 nm, 730±50 nm, 740±50 nm, 750±50 nm, 760±50 nm, 770±50 nm, 780±50 nm, 790±50 nm, 800±50 nm, 810±50 nm, 820±50 nm, 830±50 nm, 840±50 nm, 850±50 nm, 860±50 nm, 870±50 nm, 880±50 nm, 890±50 nm, 900±50 nm, 910±50 nm, 920±50 nm, 930±50 nm, 940±50 nm, 950±50 nm, 960±50 nm, 970±50 nm, 980±50 nm, 990±50 nm, 1000±50 nm, 900±100 nm, 950±100 nm, or 1000±100 nm. The aforementioned wavebands may be finely tuned such that the emitter pulses the central wavelength with a tolerance threshold of ±100 nm, ±90 nm, ±80 nm, ±70 nm, ±60 nm, ±50 nm, ±40 nm, ±30 nm, ±20 nm, ±10 nm, ±8 nm, ±6 nm, ±5 nm, ±4 nm, ±3 nm, ±2 nm, ±1 nm, and so forth. In some cases, the emitter includes a plurality of laser bundles that are each configured to pulse a particular wavelength of EMR with a tolerance threshold not greater than ±5 nm, ±4 nm, ±3 nm, or ±2 nm.

Certain multispectral wavelengths pierce through tissue and enable a medical practitioner to “see through” tissues in the foreground to identify chemical processes, structures, compounds, biological processes, and so forth that are located behind the foreground tissues. The multispectral wavelengths may be specifically selected to identify a specific disease, tissue condition, biological process, chemical process, type of tissue, and so forth that is known to have a certain spectral response.

The variable pulse cycle may include one or more emissions of EMR that are optimized for mapping imaging, which includes, for example, three-dimensional topographical mapping, depth map generation, calculating distances between objects within a scene, calculating dimensions of objects within a scene, determining whether a tool or other object approaches a threshold distance from another object, and so forth. The pulses for laser mapping imaging include EMR formed in a mapping pattern, which may include one or more of vertical hashing, horizontal hashing, a dot array, and so forth.

The controller 104 optimizes the variable pulse cycle to accommodate various imaging and video standards. In most use-cases, the system outputs a video stream comprising at least 30 frames per second (fps). The controller 104 synchronizes operations of the emitter and the image sensor to output data at a sufficient frame rate for visualizing the scene and further for processing the scene with one or more advanced visualization techniques. A user may request a real-time color video stream of the scene and may further request information based on one or more of multispectral imaging, fluorescence imaging, or laser mapping imaging (which may include topographical mapping, calculating dimensions and distances, and so forth). The controller 104 causes the image sensor to separately sense color data frames, multispectral data frames, fluorescence data frames, and mapping data frames based on the variable pulse cycle of the emitter.

In some cases, a user requests more data types than the system can accommodate while maintaining a smooth video frame rate. The system is constrained by the image sensor's ability to accumulate a sufficient amount of electromagnetic energy during each blanking period to output a data frame with sufficient exposure. In some cases, the image sensor outputs data at a rate of 60-120 fps and may specifically output data at a rate of 60 fps. In these cases, for example, the controller 104 may devote 24-30 fps to color visualization and may devote the other frames per second to one or more advanced visualization techniques.

The controller 104 calculates and adjusts the variable pulse cycle of the emitter 102 in real-time based at least in part on the known capabilities of the pixel array 125. The controller 104 may access data stored in memory indicating how long the pixel array 125 must be exposed to a certain waveband of EMR for the pixel array 125 to accumulate a sufficient amount of EMR to output a data frame with sufficient exposure. In most cases, the pixel array 125 is inherently more or less sensitive to different wavebands of EMR. Thus, the pixel array 125 may require a longer or shorter blanking period duration for some wavebands of EMR to ensure that all data frames output by the image sensor 124 comprise sufficient exposure levels.

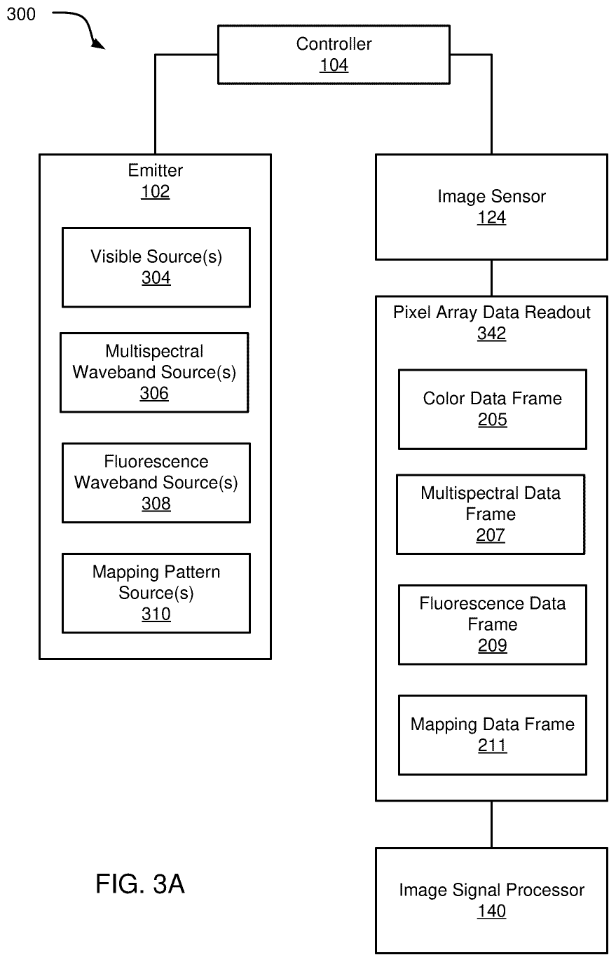

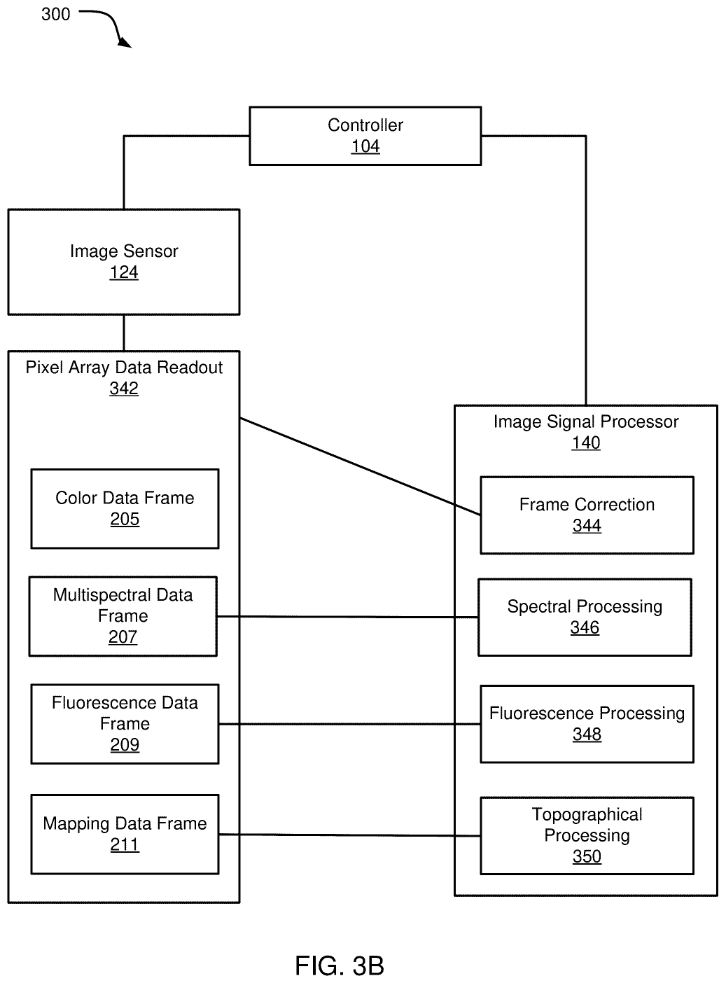

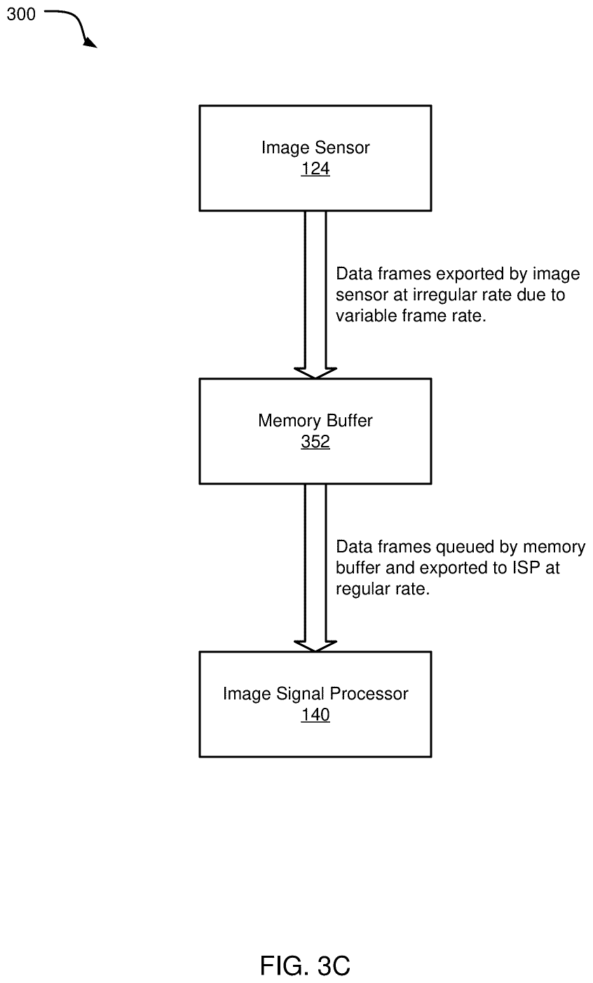

The controller 104 determines the data input requirements for various advanced visualization algorithms (see, e.g., the algorithms 346, 348, 350 first described in FIG. 3B). For example, the controller 104 may determine that certain advanced visualization algorithms do not require a data input at the same regularity as a color video stream output of 30 fps. In these cases, the controller 104 may optimize the variable pulse cycle to include white light pulses at a more frequent rate than pulses for advanced visualization such as multispectral, fluorescence, or laser mapping imaging. Additionally, the controller 104 determines whether certain algorithms may operate with lower resolution data frames that are read out by the image sensor using a pixel binning configuration. In some cases, the controller 104 ensures that all color frames provided to a user are read out in high-resolution (without pixel binning). However, some advanced visualization algorithms (see e.g., 346, 348, 350) may execute with lower resolution data frames.

The system 100 may include a plurality of image sensors 124 that may have different or identical pixel array configurations. For example, one image sensor 124 may include a monochromatic or “color agnostic” pixel array with no filters, another image sensor 124 may include a pixel array with a Bayer pattern CFA, and another image sensor 124 may include a pixel array with a different CFA. The multiple image sensors 124 may be assigned to detect EMR for a certain imaging modality, such as color imaging, multispectral imaging, fluorescence imaging, or laser mapping imaging. Further, each of the image sensors 124 may be configured to simultaneously accumulate EMR and output a data frame, such that all image sensors are capable of sensing data for all imaging modalities.

The controller 104 prioritizes certain advanced visualization techniques based on the user's ultimate goals. In some cases, the controller 104 prioritizes outputting a smooth and high-definition color video stream to the user above other advanced visualization techniques. In other cases, the controller 104 prioritizes one or more advanced visualization techniques over color visualization, and in these cases, the output color video stream may appear choppy to a human eye because the system outputs fewer than 30 fps of color imaging data.

For example, a user may indicate that a fluorescent reagent has been administered to a patient. If the fluorescent reagent is time sensitive, then the controller 104 may ensure that a sufficient ratio of frames is devoted to fluorescence imaging to ensure the user receives adequate fluorescence imaging data while the reagent remains active. In another example, a user requests a notification whenever the user's tool comes within a threshold distance of a certain tissue, such as a blood vessel, nerve fiber, cancer tissue, and so forth. In this example, the controller 104 may prioritize laser mapping visualization to constantly determine the distance between the user's tool and the surrounding structures and may further prioritize multispectral or fluorescence imaging that enables the system to identify the certain tissue. The controller 104 may further prioritize color visualization to ensure the user continues to view a color video stream of the scene.

FIGS. 3A-3C illustrate schematic diagrams of a system 300 for processing data output by an image sensor 124 comprising the pixel array 125. The system 300 includes a controller 104 in communication with each of the emitter 102 and the image sensor 124 comprising the pixel array 125. The emitter 102 includes one or more visible sources 304, multispectral waveband sources 306, fluorescence waveband sources 308, and mapping pattern sources 310 of EMR. The pixel array data readout 342 of the image sensor 124 includes one or more of the color data frames 205, multispectral data frames 207, fluorescence data frames 209, and mapping data frames 211 as discussed in connection with FIG. 2B.

As illustrated in FIG. 3B, all data read out by the pixel array may undergo frame correction 344 processing by the image signal processor 140. In various implementations, one or more of the color data frame 205, the multispectral data frame 207, the fluorescence data frame 209, and the mapping data frame 211 undergoes frame correction 344 processes. The frame correction 344 includes one or more of sensor correction, white balance, color correction, or edge enhancement.

The multispectral data frame 207 may undergo spectral processing 346 that is executed by the image signal processor 140 and/or another processor that is external to the system 300. The spectral processing 346 may include a machine learning algorithm and may be executed by a neural network configured to process the multispectral data frame 207 to identify one or more tissue structures within a scene based on whether those tissue structures emitted a spectral response.

The fluorescence data frame 209 may undergo fluorescence processing 348 that is executed by the image signal processor 140 and/or another processor that is external to the system 300. The fluorescence processing 348 may include a machine learning algorithm and may be executed by a neural network configured to process to fluorescence data frame 209 and identify an intensity map wherein a fluorescence relaxation wavelength is detected by the pixel array.

The mapping data frame 211 may undergo topographical processing 350 that is executed by the image signal processor 140 and/or another processor that is external to the system 300. The topographical processing 350 may include a machine learning algorithm and may be executed by a neural network configured to assess time-of-flight information to calculate a depth map representative of the scene. The topographical processing 350 includes calculating one or more of a three-dimensional topographical map of the scene, a dimension of one or more objects within the scene, a distance between two or more objects within the scene, a distance between a tool and a certain tissue structure within the scene, and so forth.

FIG. 3C illustrates a schematic diagram of a system 300 and process flow for managing data output at an irregular rate. The image sensor 124 operates according to a sensor cycle that includes blanking periods and readout periods. The image sensor 124 outputs a data frame at the conclusion of each readout period that includes an indication of the amount of EMR the pixel array accumulated during the preceding accumulation period or blanking period.

Each frame period in the sensor cycle is adjustable on a frame-by-frame basis to optimize the output of the image sensor and compensate for the pixel array 125 having varying degrees of sensitivity to different wavebands of EMR. The duration of each blanking period may be shortened or lengthened to customize the amount of EMR the pixel array 125 can accumulate. Additionally, the duration of each readout period may be shortened or lengthened by implementing a pixel binning configuration or causing the image sensor to read out each pixel within the pixel array 125. Thus, the image sensor 124 may output data frames at an irregular rate due to the sensor cycle comprising a variable frame rate. The system 300 includes a memory buffer 352 that receives data frames from the image sensor 124. The memory buffer 352 stores the data frames and then outputs each data frame to the image signal processor 140 at a regular rate. This enables the image signal processor 140 to process each data frame in sequence at a regular rate.

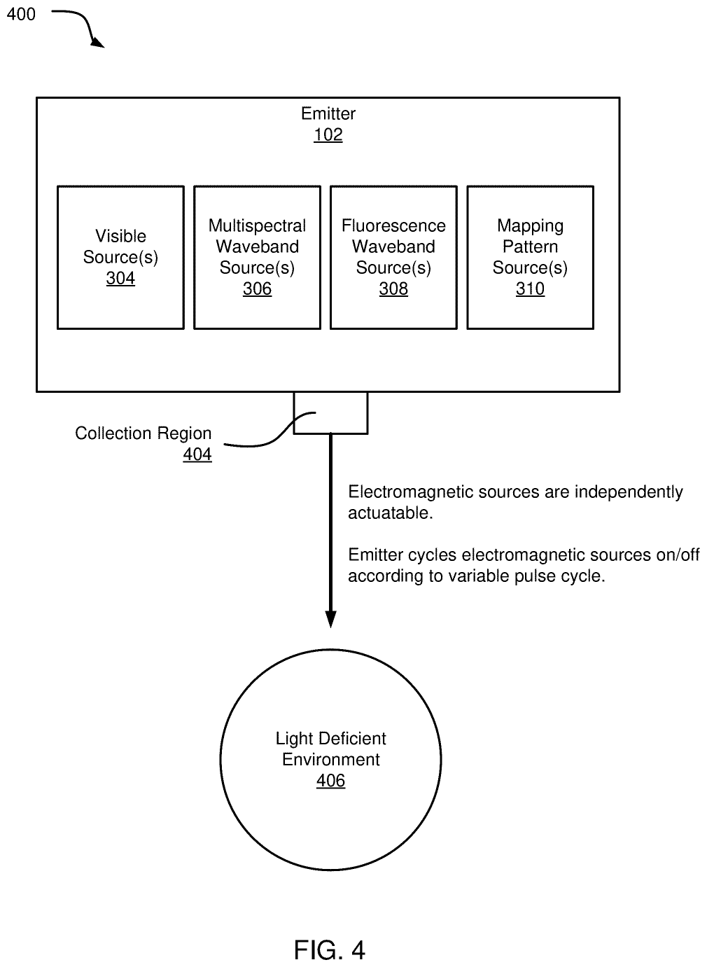

FIG. 4 is a schematic diagram of an illumination system 400 for illuminating a light deficient environment 406 such as an interior of a body cavity. In most cases, the emitter 102 is the only source of illumination within the light deficient environment 406 such that the pixel array of the image sensor does not detect any ambient light sources. The emitter 102 includes a plurality of separate and independently actuatable sources of EMR, which may include visible source(s) 304, multispectral waveband source(s) 306, fluorescence waveband source(s) 308, and mapping pattern source(s) 310. The emitter may cycle a selection of the sources on and off to pulse according to the variable pulse cycle received from the controller 104. Each of the EMR sources feeds into a collection region 404 of the emitter 102. The collection region 404 may then feed into a waveguide (see e.g., 130 in FIG. 1A) that transmits the pulsed EMR to a distal end of an endoscope within the light deficient environment 406.

The variable pulsing cycle is customizable and adjustable in real-time based on user input. The emitter 102 may instruct the individual EMR sources to pulse in any order. Additionally, the emitter 102 may adjust one or more of a duration or an intensity of each pulse of EMR. The variable pulse cycle may be optimized to sufficiently illuminate the light deficient environment 406 such that the resultant data frames read out by the pixel array 125 are within a desired exposure range (i.e., the frames are neither underexposed nor overexposed). The desired exposure range may be determined based on user input, requirements of the image signal processor 140, and/or requirements of a certain image processing algorithm (see 344, 346, 348, and 350 in FIG. 3B). The sufficient illumination of the light deficient environment 406 is dependent on the energy output of the individual EMR sources and is further dependent on the efficiency of the pixel array 125 for sensing different wavebands of EMR.

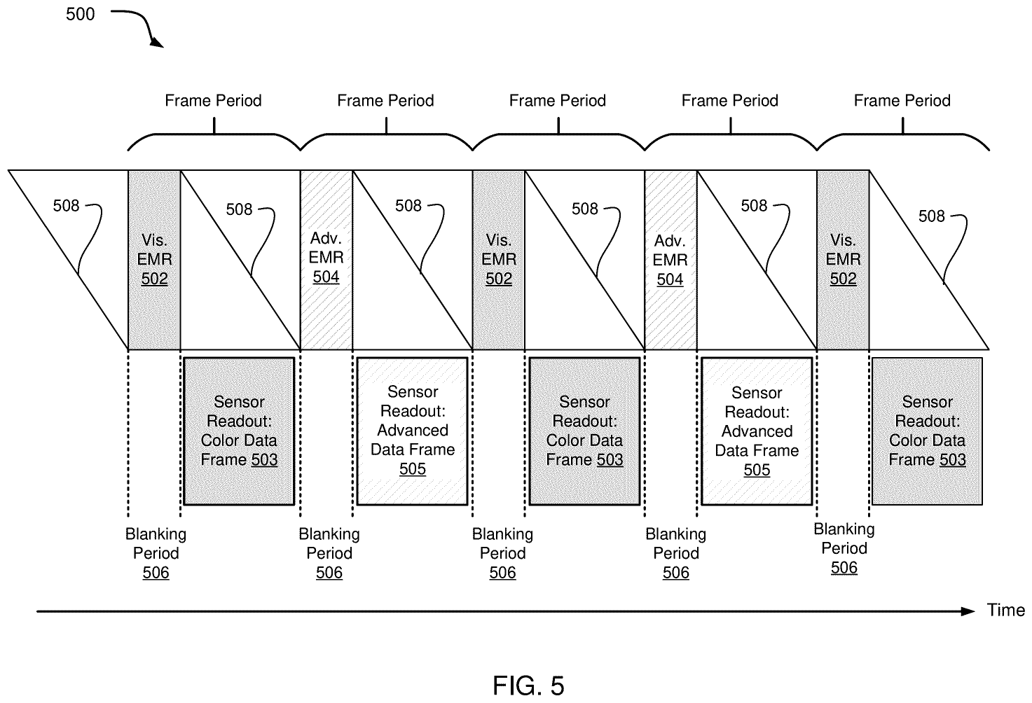

FIGS. 5-7 are schematic diagram of synchronized operational cycles 500, 600, 700 of an image sensor and an emitter. The operational cycles 500, 600, 700 may specifically be implemented when a user requests a color video stream and one or more types of advanced visualization at the same time. The user may request a display of a real-time color video stream comprising additional information obtained through one or more of multispectral visualization, fluorescence visualization, topographical mapping, or anatomical measurement scanning. The advanced visualization data may be processed by one or more algorithms, such as the algorithms 346, 348, 350 discussion in connection with FIG. 3B.

FIGS. 5-7 illustrate operational cycles 500, 600, 700 that are implemented to acquire color data frames and “advanced” data frames, wherein the advanced data frames include any of multispectral data frames 207, fluorescence data frames 209, or mapping data frames 211 as described herein. In each of FIGS. 5-7, the rolling readout sequence of the image sensor 124 is represented by diagonal lines 508, 608, 708. Additionally, illumination periods of the emitter 102 are represented by grey regions for visible EMR 502, 602, 702 and a diagonal pattern for advanced EMR 504, 604, 704. The resultant data frames are similarly represented, such that color data frames are represented by grey boxes 503, 603, 703, and advanced data frames include a diagonal pattern 505, 605, 705.

The color data frames 503, 603, 703 are sensed by the image sensor 124 in response to the emitter 102 pulsing visible EMR 502, 602, 702, which may specifically include white light. The advanced data frames 505, 605, 705 are sensed by the image sensor 124 in response to the emitter 102 emitting “advanced” EMR 504, 604, 704, which may specifically include any of multispectral 206, fluorescence 208, or mapping 210 emissions as described herein. The advanced EMR 504, 604, 704 may include a specific waveband of EMR for multispectral visualization or fluorescence visualization or may include EMR diffracted into a mapping pattern for one or more of topographical mapping, anatomical measurement, tool tracking, and so forth.

In some advanced visualization schemes described herein, the advanced imaging signal is overlayed on a color image. To achieve this, the system 100 separately acquires a color image and an advanced image. The image sensor 124 reads out a color data frame 205 in response to accumulating reflected white light. If the advanced image is a multispectral image, then the image sensor 124 reads out a multispectral data frame 207 in response to accumulating a spectral response emitted by a tissue and/or accumulating reflected EMR within a multispectral waveband. If the advanced image is a fluorescence image, then the image sensor 124 reads out a fluorescence data frame 209 in response to accumulating a fluorescence relaxation wavelength emitted by a tissue or reagent and/or accumulating reflected EMR within a fluorescence excitation waveband. In some implementations, the EMR accumulated for the advanced image (i.e., the spectral response, reflected multispectral EMR, fluorescence relaxation emission, or reflected fluorescence excitation EMR) is relatively dim when compared with the white light emission used for the color data frame. The controller (may include components of the controller 104 or the MCU 122) may adjust a pulse cycle of the emitter 102 to compensate for the relative dimness of the EMR for the advanced visualization.

The controller (may include components of the controller 104 or the MCU 122) selects an operational cycle 500, 600, 700 based on relative “strengths” of the visible EMR and the advanced EMR. In some cases, a visible EMR source is considered “stronger” based on inherent qualities of a pixel array, e.g., if a pixel array is inherently more sensitive to detecting EMR emitted by the visible EMR source, then the visible EMR source is classified as “stronger” when compared with the advanced EMR source. Further, if the pixel array is inherently less sensitive to detecting EMR emitted by the advanced EMR source, then the advanced EMR source may be classified as “weaker” when compared with the visible EMR source. It should be understood that different image sensors produced by different manufacturers, or produced with different specifications, may have different inherent sensitivities to different partitions of the electromagnetic spectrum. Thus, the relative “weaker” and “stronger” classifications for different sources of EMR may vary depending on the specifications of the image sensor being used. Additionally, a “stronger” EMR source may have a higher amplitude, greater brightness, or higher energy output when compared with a “weaker” EMR source.

The controller may adjust the pulse cycle of the emitter 102 in real-time based on the relative strengths of different EMR sources and further based on the exposure levels of resultant data frames. In an example implementation, a user requests color imaging and fluorescence imaging. The controller determines the fluorescence source is relatively weak when compared with the white light source. The fluorescence source may be relatively weak based on inherent characteristics of the pixel array. For example, the pixel array may be less sensitive to detecting the fluorescence excitation wavelength emitted by the emitter or the fluorescence relaxation wavelength emitted by a fluorescing tissue or reagent within a scene. Additionally, the fluorescence source may be relatively weak because the fluorescence emission comprises a lower amplitude, lower brightness, and/or lower energy output when compared with the white light source. In response to determining the fluorescence source is “weak” compared with the white light source, the controller may select one of the operational cycles 600, 700 that extends the illumination time for the fluorescence source. The controller may adjust and optimize the operational cycle 600, 700 in real-time to ensure that all resultant data frames are sufficiently exposed.

The image sensor 124 commonly employs a rolling shutter, in which different lines on the pixel array 125 are exposed in a sequential manner, as opposed to being exposed simultaneously. In this case, when the image sensor 124 reads out the final pixels for a first data frame, some pixels of the pixel array 125 have already begun accumulating EMR for a second data frame. In some implementations, the emitter 102 refrains from illuminating a scene during a readout period of the pixel array, and only illuminates the scene during a blanking period of the image sensor, which may be referred to as a “common exposure” period in some cases. The blanking period of the image sensor 124 (during which the pixel array 125 accumulates EMR) may have a short duration. In some cases, the pixel array 125 cannot accumulate sufficient EMR for advanced visualization during a standard blanking period.

FIG. 5 is a schematic illustration of a synchronized operational cycle 500 that may be implemented when the visible EMR 502 and the advanced EMR 504 have similar brightness levels, or when the pixel array 125 can accumulate sufficient visible EMR 502 and advanced EMR 504 during a standard blanking period 506 of the image sensor. In an example implementation, the operational cycle 500 is selected when the advanced EMR 504 includes EMR split into a mapping pattern for topographical imaging, anatomical measurement, tool tracking, and so forth. In another example implementation, the operational cycle 500 is selected when the advanced EMR 504 includes a waveband of EMR selected for multispectral or fluorescence visualization that has similar brightness levels when compared with the visible EMR 502.

In the operational cycle 500, the emitter 102 pulses visible EMR 502, and then the image sensor 124 reads out according to a rolling readout 508 scheme (see diagonal line 508 representing rolling readout of the pixel array 125) and outputs a color data frame 503. The emitter 102 pulses the advanced EMR 504 (may include any of pulsed multispectral 206, pulsed fluorescence 208, or pulsed mapping 210 as described herein), and then the image sensor 124 reads out according to a rolling readout 508 scheme and outputs an advanced data frame 505. The advanced data frame 505 may include any of a multispectral data frame 207, a fluorescence data frame 209, or a mapping data frame 211 as described herein. The operational cycle 500 limits energy accumulation to the blanking period 506 of the image sensor 124, such that the pixel array 125 does not accumulate EMR when the image sensor is executing the rolling readout 506 scheme.

The operational cycle 500 may result in the “cleanest” data frames, wherein all pixels within the pixel array 125 are exposed to EMR for the same duration of time, because the pixel array 125 accumulates EMR only during the blanking periods 506. The resultant data frames 503, 505 may be free from image artifacts resulting from some pixels accumulating EMR for a longer duration of time than other pixels. However, the operational cycle 500 may be insufficient if the pixel array 125 cannot accumulate sufficient advanced EMR 504 during the blanking period 506. The operational cycle 500 leaves much of the image sensor's available exposure time unused.

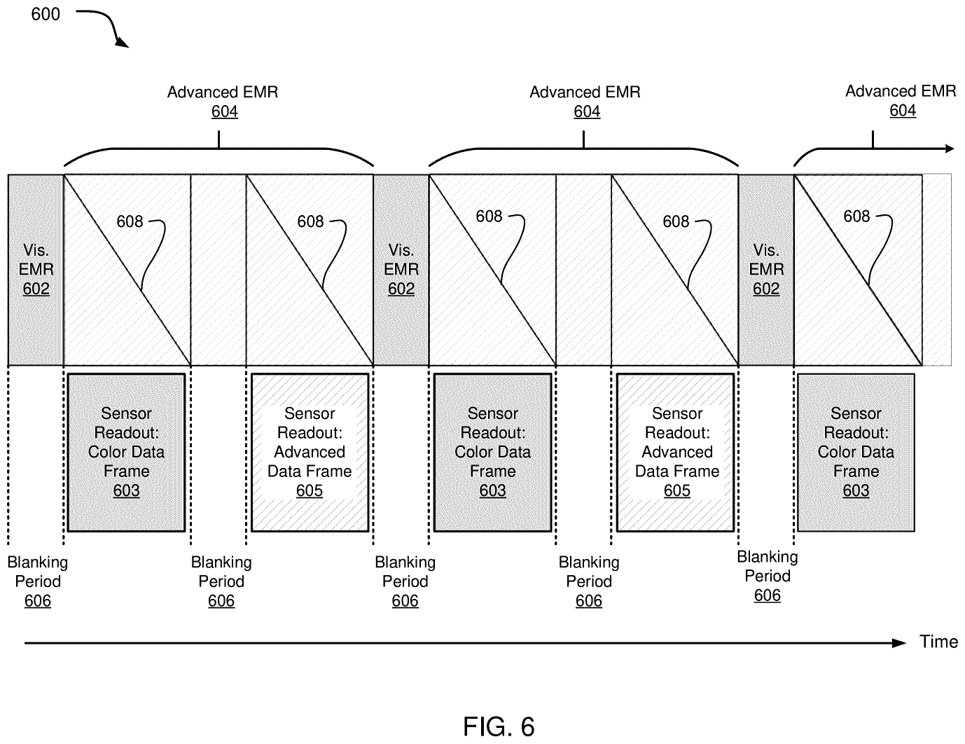

FIG. 6 is a schematic illustration of a synchronized operational cycle 600 that may be implemented when the visible EMR 602 and advanced EMR 604 have different brightness levels, or when the pixel array 125 does not accumulate sufficient advanced EMR 604 during a single blanking period 606. In an example implementation, the operational cycle 600 is selected when the advanced EMR 604 includes a waveband of EMR selected for fluorescence visualization that is relatively dim when compared with the visible EMR 602. The operational cycle 600 may specifically be implemented when the advanced EMR 604 includes a near infrared (NIR) waveband of the electromagnetic spectrum.

In the operational cycle 600, the emitter 102 pulses the advanced EMR 604 for a longer duration to enable the pixel array 125 to accumulate the advanced EMR 604 for a longer duration of time. This seeks to compensate for the brightness disparity between the visible EMR 602 and the advanced EMR 604 to ensure that each of the color data frames 603 and the advanced data frames 605 comprise sufficient data. In FIG. 6, the emissions of the advanced EMR 604 are represented by regions including a diagonal line pattern, and the resultant advanced data frames 605 are represented with the same pattern. As shown in FIG. 6, the emitter 102 pulses the advanced EMR 604 continuously throughout the rolling readout 608 for the color data frame 603, the blanking period 606 between the color data frame 603 and the advanced data frame 605, and still during the rolling readout 608 for the advanced data frame 605. The advanced EMR 604 is turned off only during the blanking period 606 when the visible EMR 602 is pulsed.

The operational cycle 600 enables the pixel array 125 to accumulate the advanced EMR 604 for a longer duration of time. This may be important if the pixel array 125 is inherently inefficient at accumulating the advanced EMR 604 and/or if the advanced EMR 604 is relatively dim when compared with the visible EMR 602.

In the operational cycle 600, the color data frames 603 are partially exposed to the advanced EMR 604 where the data frames overlap. Thus, the resulting color data frames 603 include the sum of the accumulated energy from the visible EMR 602 and some of the advanced EMR 604. However, this “overflow” of advanced EMR 604 into the color data frame 603 does not impact integrity of the final overlay for several reasons. Namely, the advanced EMR 604 signal is typically very weak when compared with the visible EMR 602, and thus its contribution in the color data frame 603 is negligible in most cases. Additionally, where the advanced EMR 604 signal is high or comparable to the visible EMR 602 signal, the effect is masked by an overlay display when the color data frame 603 and the advanced data frame 605 are combined. Additionally, the two components (i.e., the reflectance signal from the visible EMR 602 and the signal from the advanced EMR 604) may be separated with additional image processing.

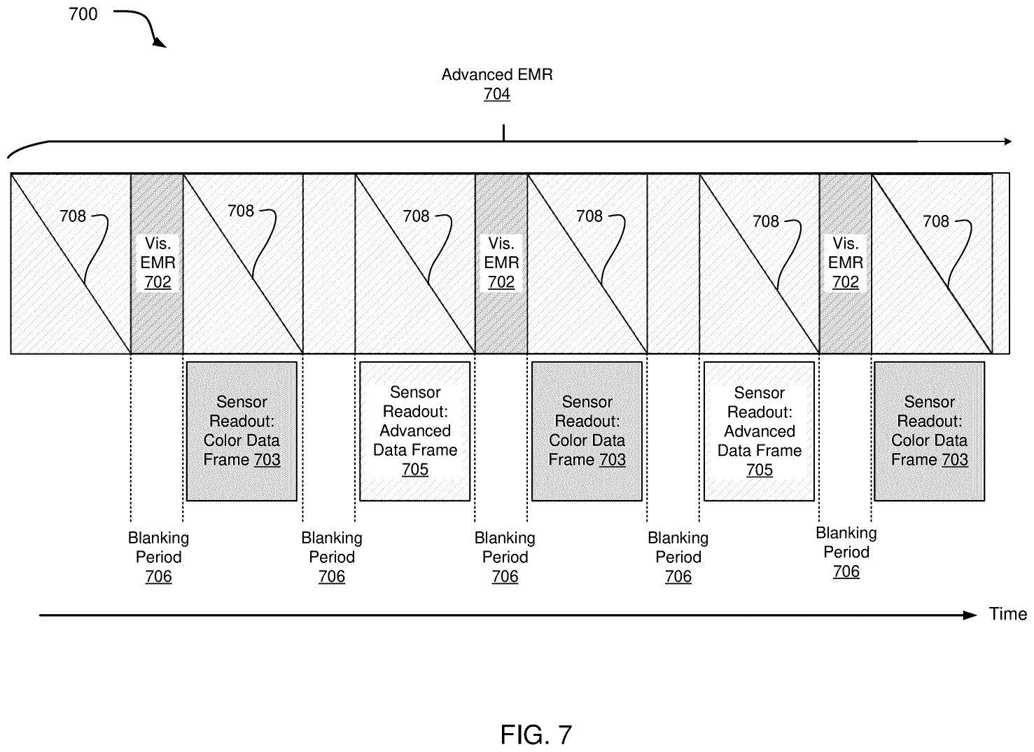

FIG. 7 is a schematic illustration of a synchronized operational cycle 700 that may be implemented when the visible EMR 702 and advanced EMR 704 have different brightness levels, or when the pixel array 125 does not accumulate sufficient advanced EMR 704 during a single blanking period 706. In an example implementation, the operational cycle 700 is selected when the advanced EMR 704 includes a waveband of EMR selected for fluorescence visualization that is relatively dim when compared with the visible EMR 702. The operational cycle 700 may specifically be implemented when the advanced EMR 704 includes a near infrared (NIR) waveband of the electromagnetic spectrum.

In the operational cycle 700, the emitter 102 constantly emits the advanced EMR 704, rather than cycling the advanced EMR 704 on and off. The emitter 102 emits the advanced EMR 702 during the rolling readout 708 for the color data frame 703, during all blanking periods 706, and during the rolling readout 708 for the advanced data frames 705.

The operational cycles 600, 700 allow for a significantly longer exposure time for the advanced data frame. This greatly increases sensitivity of the advanced data frame and makes efficient use of the image sensor's exposure, because the entire rolling readout shutter period is used to accumulate EMR, as opposed to only accumulating EMR during the blanking period.

The resultant data frames from any of the operational cycles 500, 600, 700 may be overlaid to output a color image combined with advanced visualization data. In some cases, the location of a tissue or region within a scene is identified with multispectral or fluorescence visualization, and in these cases, the identified tissue or region may be displayed with a false color as an overlay on a color image depicting the scene. In an example implementation, the user is presented with a color video stream of a scene, and a desired tissue structure (e.g., cancer cells, nerve tissue, blood, and so forth) is highlighted with a single color that may be selected by the user. In another implementation, the user is presented with a color video stream of a scene, and a desired tissue structure is highlighted with an intensity map indicating the relative likelihood of where the tissue structure is located. The intensity map possesses similarities to a temperature-based heat map but will instead indicate the relative intensities of the spectral or fluorescence signal accumulated by the pixel array 125.

The operational cycles 600, 700 may specifically be implemented when the advanced EMR 604, 704 is a fluorescence excitation wavelength within the infrared or near infrared range of the electromagnetic spectrum. The operational cycles 600, 700 may also be deployed when pulsing wavebands within the visible and long red ranges of the electromagnetic spectrum. Infrared and near infrared EMR is dimmer than white light, and thus, the image sensor may struggle to accumulate sufficient energy to read out a fluorescence data frame in response to being exposed with the fluorescence excitation wavelength.

The controller optimizes the pulse cycle of the emitter 102 and the sensor cycle of the image sensor 124 based on user input, pulse type (e.g., white light, multispectral waveband, fluorescence waveband, or EMR split into a mapping pattern), desired exposure of resultant data frames, desired frame rate, and acceptable resolution of resultant data frames. The systems, methods, and devices described herein interlace a color video data stream with one or more advanced visualization data streams. Each of these data streams may be sensed by a single image sensor 124, multiple image sensors 124 acting in concert, or multiple image sensor 124 acting in sequence.

The controller may ensure that each resultant data frame comprises exposure (brightness) levels within a threshold range. The threshold exposure range may be different based on the frame type. For example, color data frames may have a different threshold exposure range than advanced data frames because the color data frames will be viewed by a user on a display while the advanced data frames are likely to be assessed by an algorithm (see, e.g., algorithms 346, 348, 350 discussed in connection with FIG. 3B). The various algorithms 346, 348, 350 may have different exposure requirements to successfully make determinations based on the data frame. Thus, the controller may need to extend the illumination time for an advanced data frame to compensate for the relative weakness of the advanced EMR source, and further to ensure the resultant data frame can be assessed by a corresponding algorithm.

The controller may adjust durations for the readout periods and blanking periods in real-time based on user input and resultant data frames being processed through a feedback loop. For example, if the controller determines that a previous multispectral data frame could not be assessed by the spectral processing 344 algorithm because the multispectral data frame was underexposed, then the controller may lengthen the multispectral illumination time for subsequent multispectral frames. The controller may switch between any of the operational cycles 500, 600, 700 depending on the EMR sources being used, user feedback, resultant data frames, and so forth. The controller may generate a custom operational cycle that comprises components of any of the operational cycles 500, 600, 700.

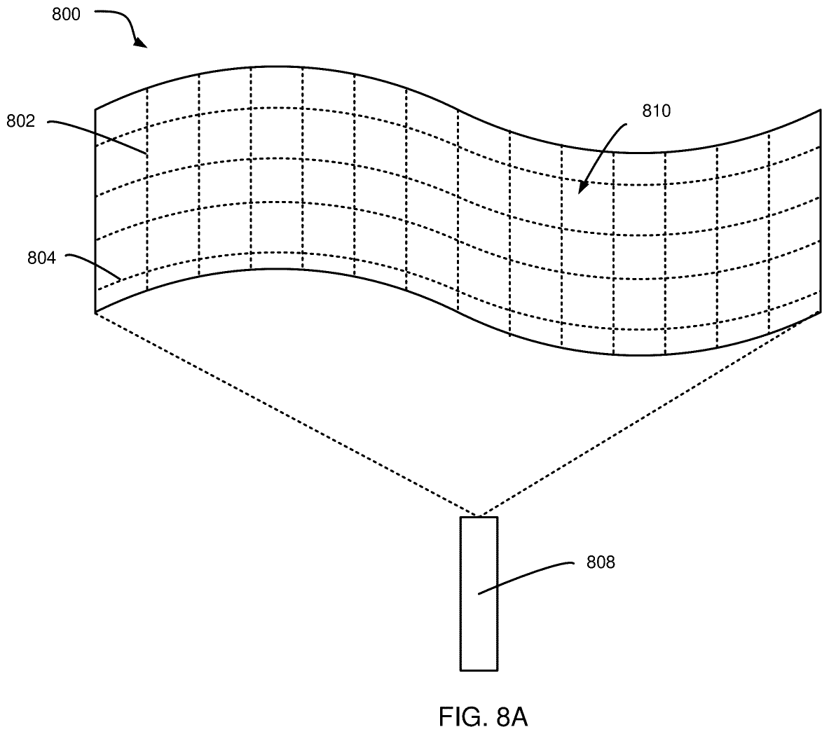

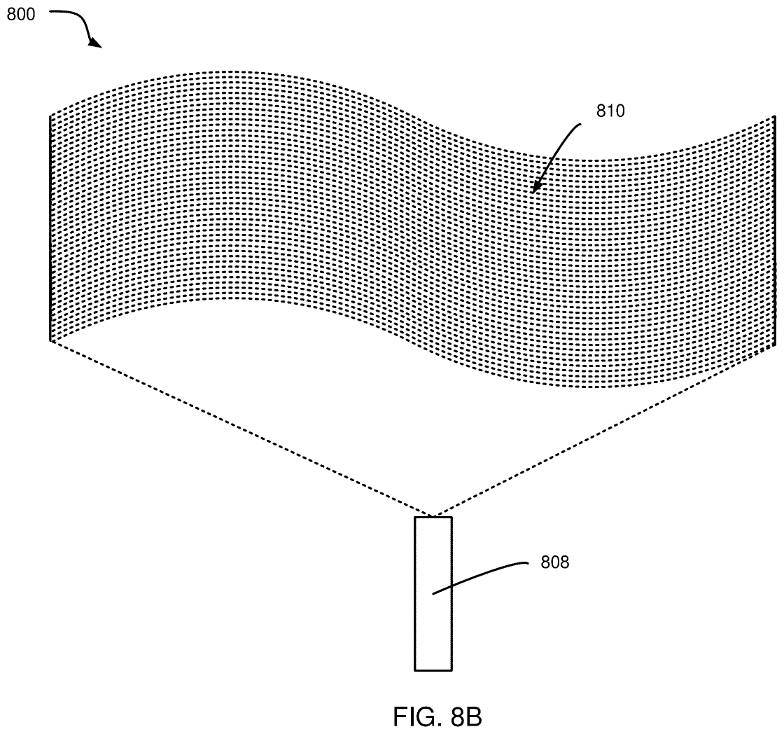

FIGS. 8A and 8B illustrate schematic diagrams of a system 800 for emitting EMR in a mapping pattern 210. The emitter 102 may pulse the mapping pattern 210 using a low mode laser light that is diffracted to generate the applicable mapping pattern 210. The mapping pattern 210 reflects off tissue in a way that depends on the wavelength of the EMR and the specific anatomical features of the tissue.

The example mapping pattern 210 illustrated in FIG. 8A is a grid array comprising vertical hashing 802 and horizontal hashing 804. The example mapping pattern 210 illustrated in FIG. 8B is a dot array. The mapping pattern 210 may include any suitable array for mapping a surface, including, for example, a raster grid of discrete points, an occupancy grid map, a dot array, vertical hashing, horizontal hashing, and so forth. The mapping pattern 210 is emitted by an illumination source 808, which may originate with an EMR source within the emitter 102 and terminate with an endoscope 110 or tool 108.

As discussed in connection with FIGS. 1A-1C, the distal end of an endoscope 110 may include one or more waveguides 130 that emit EMR that originated at the emitter 102. The mapping pattern 210 may be emitted from these waveguides 130 and projected on to a surface of a tissue. Additionally, one or more tools 108 within a scene may include a waveguide 131 that terminates at a distal end of the tool 108 and/or a side of the tool 108 as shown in FIG. 1A. This waveguide 131 may also emit the mapping pattern 210 on to a surface of a tissue within a scene. In some cases, the tool 108 and the endoscope 110 emit the mapping pattern 210 in concert, and the resultant data frames captured by the image sensor 124 comprise data for tracking the location of the tool 108 within the scene relative to the endoscope 110 or other tools 108.

The emitter 102 may pulse the mapping pattern 210 at any suitable wavelength of EMR, including, for example, ultraviolet light, visible, light, and/or infrared or near infrared light. The surface and/or objects within the environment may be mapped and tracked at very high resolution and with very high accuracy and precision.

The mapping pattern 210 is selected for the desired anatomical measurement scheme, such as three-dimensional topographical mapping, measuring distances and dimensions within a scene, tracking a relative position of a tool 108 within a scene, and so forth. The image sensor 124 detects reflected EMR and outputs a mapping data frame 211 in response to the emitter 102 pulsing the mapping pattern 210. The resultant mapping data frame 211 is provided to a topographical processing 350 algorithm that is trained to calculate one or more of a three-dimensional topographical map of a scene, a distance between two or more objects within the scene, a dimension of an object within the scene, a relative distance between a tool and another object within the scene, and so forth.

The emitter 102 may pulse the mapping pattern 210 at a sufficient speed such that the mapping pattern 210 is not visible to a user. In various implementations, it may be distracting to a user to see the mapping pattern 210 during an endoscopic visualization procedure. In some cases, a rendering of the mapping pattern 210 may be overlaid on a color video stream to provide further context to a user visualizing the scene. The user may further request to view real-time measurements of objects within the scene and real-time proximity alerts when a tool approaches a critical structure such as a blood vessel, nerve fiber, cancer tissue, and so forth. The accuracy of the measurements may be accurate to less than one millimeter.

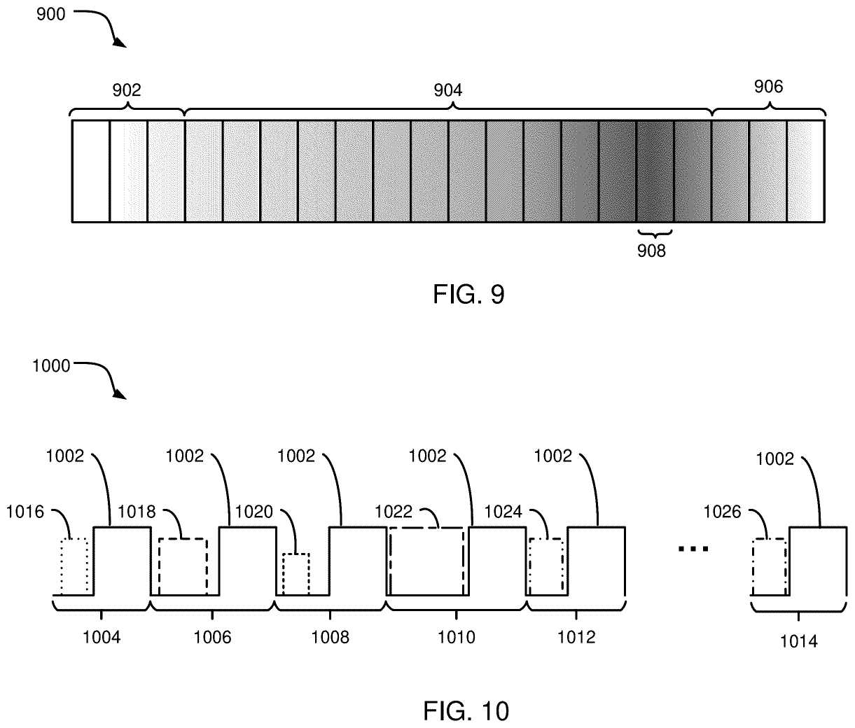

FIG. 9 illustrates a portion of the electromagnetic spectrum 900 divided into twenty different wavebands. The number of wavebands is illustrative only. In at least one embodiment, the spectrum 900 may be divided into hundreds of wavebands. The spectrum 900 may extend from the infrared spectrum 902, through the visible spectrum 904, and into the ultraviolet spectrum 906. Each waveband may be defined by an upper wavelength and a lower wavelength.

Multispectral imaging incudes imaging information from across the electromagnetic spectrum 900. A multispectral pulse of EMR may include a plurality of sub-pulses spanning one or more portions of the electromagnetic spectrum 900 or the entirety of the electromagnetic spectrum 900. A multispectral pulse of EMR may include a single partition of wavelengths of EMR. A resulting multispectral data frame includes information sensed by the pixel array subsequent to a multispectral pulse of EMR. Therefore, a multispectral data frame may include data for any suitable partition of the electromagnetic spectrum 900 and may include multiple data frames for multiple partitions of the electromagnetic spectrum 900.

The emitter 102 may include any number of multispectral EMR sources as needed depending on the implementation. In one embodiment, each multispectral EMR source covers a spectrum covering 40 nanometers. For example, one multispectral EMR source may emit EMR within a waveband from 500 nm to 540 nm while another multispectral EMR source may emit EMR within a waveband from 540 nm to 580 nm. In another embodiment, multispectral EMR sources may cover other sizes of wavebands, depending on the types of EMR sources available or the imaging needs. Each multispectral EMR source may cover a different slice of the electromagnetic spectrum 900 ranging from far infrared, mid infrared, near infrared, visible light, near ultraviolet and/or extreme ultraviolet. In some cases, a plurality of multispectral EMR sources of the same type or wavelength may be included to provide sufficient output power for imaging. The number of multispectral EMR sources needed for a specific waveband may depend on the sensitivity of a pixel array 125 to the waveband and/or the power output capability of EMR sources in that waveband.

The waveband widths and coverage provided by the EMR sources may be selected to provide any desired combination of spectrums. For example, contiguous coverage of a spectrum 900 using very small waveband widths (e.g., 10 nm or less) may allow for highly selective multispectral and/or fluorescence imaging. The waveband widths allow for selectively emitting the excitation wavelength(s) for one or more particular fluorescent reagents. Additionally, the waveband widths may allow for selectively emitting certain partitions of multispectral EMR for identifying specific structures, chemical processes, tissues, biological processes, and so forth. Because the wavelengths come from EMR sources which can be selectively activated, extreme flexibility for fluorescing one or more specific fluorescent reagents during an examination can be achieved. Additionally, extreme flexibility for identifying one or more objects or processes by way of multispectral imaging can be achieved. Thus, much more fluorescence and/or multispectral information may be achieved in less time and within a single examination which would have required multiple examinations, delays because of the administration of dyes or stains, or the like.

FIG. 10 is a schematic diagram illustrating a timing diagram 1000 for emission and readout for generating an image. The solid line represents readout (peaks 1002) and blanking periods (valleys) for capturing a series of data frames 1004-1014. The series of data frames 1004-1014 may include a repeating series of data frames which may be used for generating mapping, multispectral, and/or fluorescence data that may be overlaid on an RGB video stream. The series of data frames include a first data frame 1004, a second data frame 1006, a third data frame 1008, a fourth data frame 1010, a fifth data frame 1012, and an Nth data frame 1026.

In one embodiment, each data frame is generated based on at least one pulse of EMR. The pulse of EMR is reflected and detected by the pixel array 125 and then read out in a subsequent readout (1002). Thus, each blanking period and readout results in a data frame for a specific waveband of EMR. For example, the first data frame 1004 may be generated based on a waveband of a first one or more pulses 1016, a second data frame 1006 may be generated based on a waveband of a second one or more pulses 1018, a third data frame 1008 may be generated based on a waveband of a third one or more pulses 1020, a fourth data frame 1010 may be generated based on a waveband of a fourth one or more pulses 1022, a fifth data frame 1012 may be generated based on a waveband of a fifth one or more pulses 1024, and an Nth data frame 1026 may be generated based on a waveband of an Nth one or more pulses 1026.

The pulses 1016-1026 may include energy from a single EMR source or from a combination of two or more EMR sources. For example, the waveband included in a single readout period or within the plurality of data frames 1004-1014 may be selected for a desired examination or detection of a specific tissue or condition. According to one embodiment, one or more pulses may include visible spectrum light for generating an RGB or black and white image while one or more additional pulses are emitted to sense a spectral response to a multispectral wavelength of EMR.

The pulses 1016-1026 are emitted according to a variable pulse cycle determined by the controller 104. For example, pulse 1016 may include a white light, pulse 1018 may include a multispectral waveband, pulse 1020 may include a white light, pulse 1022 may include a fluorescence waveband, pulse 1024 may include white light, and so forth.

The plurality of frames 1004-1014 are shown having varying lengths in readout periods and pulses having different lengths or intensities. The blanking period, pulse length or intensity, or the like may be selected based on the sensitivity of a monochromatic sensor to the specific wavelength, the power output capability of the EMR source(s), and/or the carrying capacity of the waveguide.

In one embodiment, dual image sensors may be used to obtain three-dimensional images or video feeds. A three-dimensional examination may allow for improved understanding of a three-dimensional structure of the examined region as well as a mapping of the different tissue or material types within the region.

In an example implementation, a patient is imaged with an endoscopic imaging system to identify quantitative diagnostic information about the patient's tissue pathology. In the example, the patient is suspected or known to suffer from a disease that can be tracked with multispectral imaging to observe the progression of the disease in the patient's tissue. The endoscopic imaging system pulses white light to generate an RGB video stream of the interior of the patient's body. Additionally, the endoscopic imaging system pulses one or more multispectral wavebands of light that permit the system to “see through” some tissues and generate imaging of the tissue affected by the disease. The endoscopic imaging system senses the reflected multispectral EMR to generate multispectral imaging data of the diseased tissue, and thereby identifies the location of the diseased tissue within the patient's body. The endoscopic imaging system may further emit a mapping pulsing scheme for generating a three-dimensional topographical map of the scene and calculating dimensions of objects within the scene. The location of the diseased tissue (as identified by the multispectral imaging data) may be combined with the topographical map and dimensions information that is calculated with the mapping data. Therefore, the precise location, size, dimensions, and topology of the diseased tissue can be identified. This information may be provided to a medical practitioner to aid in excising, imaging, or studying the diseased tissue. Additionally, this information may be provided to a robotic surgical system to enable the surgical system to excise the diseased tissue.

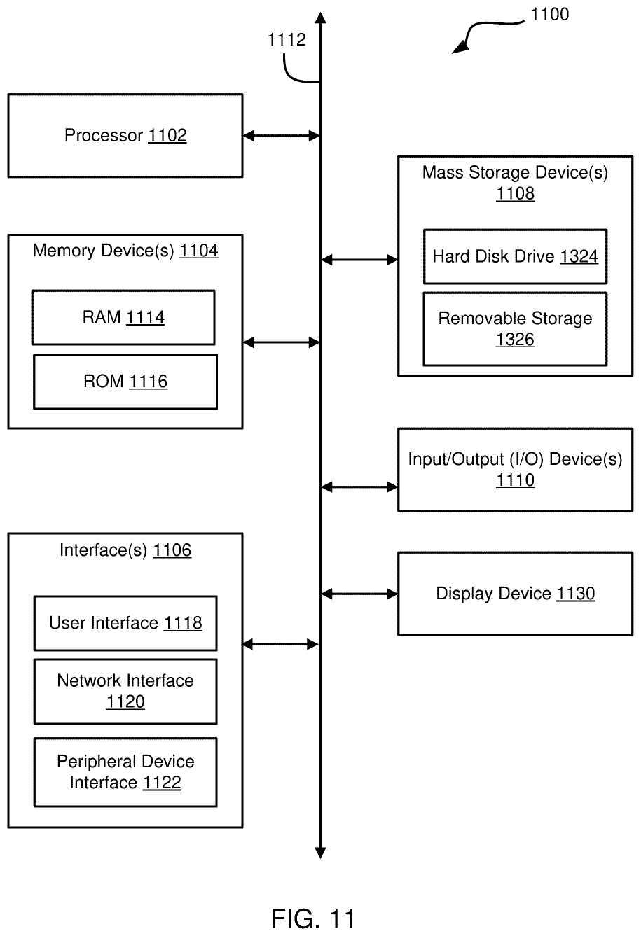

FIG. 11 illustrates a schematic block diagram of an example computing device 1100. The computing device 1100 may be used to perform various procedures, such as those discussed herein. The computing device 1100 can perform various monitoring functions as discussed herein, and can execute one or more application programs, such as the application programs or functionality described herein. The computing device 1100 can be any of a wide variety of computing devices, such as a desktop computer, in-dash computer, vehicle control system, a notebook computer, a server computer, a handheld computer, tablet computer and the like.

The computing device 1100 includes one or more processor(s) 1104, one or more memory device(s) 1104, one or more interface(s) 1106, one or more mass storage device(s) 1108, one or more Input/output (I/O) device(s) 1110, and a display device 1130 all of which are coupled to a bus 1112. Processor(s) 1104 include one or more processors or controllers that execute instructions stored in memory device(s) 1104 and/or mass storage device(s) 1108. Processor(s) 1104 may also include several types of computer-readable media, such as cache memory.

Memory device(s) 1104 include various computer-readable media, such as volatile memory (e.g., random access memory (RAM) 1114) and/or nonvolatile memory (e.g., read-only memory (ROM) 1116). Memory device(s) 1104 may also include rewritable ROM, such as Flash memory.

Mass storage device(s) 1108 include various computer readable media, such as magnetic tapes, magnetic disks, optical disks, solid-state memory (e.g., Flash memory), and so forth. As shown in FIG. 11, a particular mass storage device 1108 is a hard disk drive 1124. Various drives may also be included in mass storage device(s) 1108 to enable reading from and/or writing to the various computer readable media. Mass storage device(s) 1108 include removable media 1126 and/or non-removable media.

I/O device(s) 1110 include various devices that allow data and/or other information to be input to or retrieved from computing device 1100. Example I/O device(s) 1110 include cursor control devices, keyboards, keypads, microphones, monitors or other display devices, speakers, printers, network interface cards, modems, and the like.

Display device 1130 includes any type of device capable of displaying information to one or more users of computing device 1100. Examples of display device 1130 include a monitor, display terminal, video projection device, and the like.

Interface(s) 1106 include various interfaces that allow computing device 1100 to interact with other systems, devices, or computing environments. Example interface(s) 1106 may include any number of different network interfaces 1120, such as interfaces to local area networks (LANs), wide area networks (WANs), wireless networks, and the Internet. Other interface(s) include user interface 1118 and peripheral device interface 1122. The interface(s) 1106 may also include one or more user interface elements 1118. The interface(s) 1106 may also include one or more peripheral interfaces such as interfaces for printers, pointing devices (mice, track pad, or any suitable user interface now known to those of ordinary skill in the field, or later discovered), keyboards, and the like.

Bus 1112 allows processor(s) 1104, memory device(s) 1104, interface(s) 1106, mass storage device(s) 1108, and I/O device(s) 1110 to communicate with one another, as well as other devices or components coupled to bus 1112. Bus 1112 represents one or more of several types of bus structures, such as a system bus, PCI bus, IEEE bus, USB bus, and so forth.

For purposes of illustration, programs and other executable program components are shown herein as discrete blocks, such as block 302 for example, although it is understood that such programs and components may reside at various times in different storage components of computing device 1100 and are executed by processor(s) 1102. Alternatively, the systems and procedures described herein, including programs or other executable program components, can be implemented in hardware, or a combination of hardware, software, and/or firmware. For example, one or more application specific integrated circuits (ASICs) can be programmed to carry out one or more of the systems and procedures described herein.

### EXAMPLES

The following examples pertain to preferred features of further embodiments:

Example 1 is a system. The system includes an image sensor comprising a pixel array. The system includes an emitter comprising a plurality of electromagnetic radiation (EMR) sources that include a stronger EMR source and a weaker EMR source. The pixel array is relatively inefficient at accumulating EMR emitted by the weaker EMR source when compared with EMR emitted by the stronger EMR source. The emitter cycles at least a portion of the plurality of EMR sources according to a pulse cycle that includes a plurality of pulsed emissions by the stronger EMR source and at least one emission by the weaker EMR source. The at least one emission by the weaker EMR source overlaps a readout period of the image sensor when the image sensor reads out a data frame corresponding with an emission by the stronger EMR source.

Example 2 is a system as in Example 1, wherein the weaker EMR source emits EMR within one or more of: a near infrared waveband of the electromagnetic spectrum; or an infrared waveband of the electromagnetic spectrum

Example 3 is a system as in any of Examples 1-2, wherein the stronger EMR source emits one or more of: white light; or a partition of EMR from a visible waveband of the electromagnetic spectrum.

Example 4 is a system as in any of Examples 1-3, wherein the EMR emitted by the weaker EMR source comprises a shorter amplitude when compared with the EMR emitted by the stronger EMR source.

Example 5 is a system as in any of Examples 1-4, wherein the EMR emitted by the weaker EMR source comprises less energy when compared with the EMR emitted by the stronger EMR source.

Example 6 is a system as in any of Examples 1-5, wherein the EMR emitted by the weaker EMR source is dimmer than the EMR emitted by the stronger EMR source.

Example 7 is a system as in any of Examples 1-6, wherein the stronger EMR source and the weaker EMR source emit different wavebands of the electromagnetic spectrum, and wherein the pixel array is inherently less efficient at accumulating a waveband of EMR emitted by the weaker EMR source when compared with a waveband of EMR emitted by the stronger EMR source.

Example 8 is a system as in any of Examples 1-7, wherein the stronger EMR source is a white light source, and wherein the data frame corresponding with the emission by the stronger EMR source is a color data frame.

Example 9 is a system as in any of Examples 1-8, wherein the weaker EMR source is a fluorescence source configured to emit a fluorescence excitation wavelength of EMR, and wherein: the fluorescence excitation wavelength of EMR is selected to cause one or more of a reagent or a tissue to fluoresce; the pixel array accumulates a fluorescence relaxation wavelength of EMR emitted by the one or more of the reagent or the tissue; and the image sensor reads out a fluorescence data frame during a rolling readout sequence assigned to the weaker EMR source.

Example 10 is a system as in any of Examples 1-9, wherein the stronger EMR source is cycled off during a blanking period of the image sensor immediately preceding the rolling readout sequence assigned to the weaker EMR source.

Example 11 is a system as in any of Examples 1-10, wherein the image sensor reads out a color data frame and an advanced data frame, and wherein: the pixel array accumulates EMR resulting from an emission by the stronger EMR source to output the color data frame; and the pixel array accumulates EMR resulting from an emission by the weaker EMR source to output the advanced data frame.

Example 12 is a system as in any of Examples 1-11, wherein the advanced data frame comprises one or more of: a fluorescence data frame corresponding with the pixel array accumulating one or more of a fluorescence excitation wavelength of EMR or a fluorescence relaxation wavelength of EMR; or a multispectral data frame corresponding with the pixel array accumulating one or more of a multispectral wavelength of EMR or a spectral response emission of EMR.

Example 13 is a system as in any of Examples 1-12, wherein the pulse cycle is such that the stronger EMR source is cycled on during a blanking period immediately preceding readout of the color data frame; and wherein the stronger EMR source is cycled off during a blanking period immediately preceding readout of the advanced data frame.

Example 14 is a system as in any of Examples 1-13, wherein the pulse cycle is such that the weaker EMR source is cycled on during each of: the blanking period immediately preceding the readout of the advanced data frame; and a rolling readout sequence when the image sensor is reading out the color data frame.

Example 15 is a system as in any of Examples 1-14, wherein the pulse cycle is such that the weaker EMR source is further cycled on during a rolling readout sequence when the image sensor is reading out the advanced data frame.

Example 16 is a system as in any of Examples 1-15, wherein the pulse cycle is such that the weaker EMR source is cycled on during each of: a blanking period immediately preceding readout of the color data frame; and a blanking period immediately preceding readout of the advanced data frame.

Example 17 is a system as in any of Examples 1-16, wherein the pulse cycle is such that: the stronger EMR source is cycled on during a blanking period immediately preceding readout of the color data frame; the stronger EMR source is cycled off during a rolling readout sequence when the image sensor is reading out the color data frame; the stronger EMR source is cycled off during a blanking period immediately preceding readout of the advanced data frame; and the stronger EMR source is cycled off during a rolling readout sequence when the image sensor is reading out the advanced data frame.

Example 18 is a system as in any of Examples 1-17, wherein the pulse cycle is such that: the weaker EMR source is cycled off during the blanking period immediately preceding the readout of the color data frame; the weaker EMR source is cycled on during the rolling readout sequence when the image sensor is reading out the color data frame; the weaker EMR source is cycled on during the blanking period immediately preceding the readout of the advanced data frame; and the weaker EMR source is cycled on during the rolling readout sequence when the image sensor is reading out the advanced data frame.

Example 19 is a system as in any of Examples 1-18, wherein the pulse cycle is such that the weaker EMR source is cycled on continuously.

Example 20 is a system as in any of Examples 1-19, wherein information from the color data frame and the advanced data frame are combined to generate an overlay frame, wherein the overlay frame comprises a color image depicting a scene and further comprises a false color overlay depicting information determined based on the advanced data frame.

Example 21 is a system. The system includes an image sensor comprising a pixel array. The system includes an emitter comprising a plurality of sources of electromagnetic radiation, wherein the plurality of sources comprises a first source and a second source. The emitter cycles at least a portion of the plurality of sources according to a pulse cycle comprising: a plurality of pulsed emissions by the first source; and a plurality of pulsed emissions by the second source. The system is such that at least a portion of the plurality of pulsed emissions by the second source overlap a readout period of the image sensor when the image sensor reads out a data frame corresponding with an emission by the first source. The system is such that the second source cycles off during at least a portion of the plurality of pulsed emissions by the first source.

Example 22 is a system as in Example 21, wherein the first source is a visible source that pulses electromagnetic radiation within a visible waveband of the electromagnetic spectrum; wherein the data frame corresponding with the emission by the first source is a color data frame; wherein the pixel array accumulates reflected electromagnetic radiation during a blanking period when the emitter pulses the visible source; and wherein the image sensor reads out the color data frame during a readout period immediately subsequent to the blanking period when the emitter pulses the visible source.

Example 23 is a system as in any of Examples 21-22, wherein the second source is an excitation source configured to emit a fluorescence excitation wavelength of electromagnetic radiation; wherein the fluorescence excitation wavelength of electromagnetic radiation is selected to cause one or more of a reagent or a tissue to fluoresce; wherein the pixel array accumulates a fluorescence relaxation wavelength of electromagnetic radiation emitted by the one or more of the reagent or the tissue; and wherein the image sensor reads out a fluorescence data frame during a readout period corresponding with the second source.

Example 24 is a system as in any of Examples 21-23, wherein the second source is cycled on during each of: the readout period when the image sensor reads out the color data frame; and a blanking period immediately subsequent to the image sensor reading out the color data frame.

Example 25 is a system as in any of Examples 21-24, wherein the second source is further cycled on during the readout period corresponding with the second source wherein the image sensor reads out the fluorescence data frame; and wherein the second source is cycled on continuously throughout each of: the readout period when the image sensor reads out the color data frame; the blanking period immediately subsequent to the image sensor reading out the color data frame; and the readout period wherein the image sensor reads out the fluorescence data frame.

Example 26 is a system as in any of Examples 21-25, wherein the emitter cycles off the first source during each of: the readout period when the image sensor reads out the color data frame; the blanking period immediately subsequent to the image sensor reading out the color data frame; and the readout period wherein the image sensor reads out the fluorescence data frame.

Example 27 is a system as in any of Examples 21-26, wherein the emitter cycles the first source and the second source on and off according to the pulse cycle such that the plurality of pulsed emissions by the first source do not overlap with the plurality of pulsed emissions by the second source.

Example 28 is a system as in any of Examples 21-27, further comprising: an endoscope, wherein the image sensor is disposed within a distal region of the endoscope; and a prism disposed within the distal region of the endoscope configured to reflect electromagnetic radiation on to the image sensor; wherein a planar side of the pixel array is oriented parallel to a longitudinal axis of the endoscope.

Example 29 is a system as in any of Examples 21-28, wherein the pixel array is relatively inefficient at accumulating electromagnetic radiation emitted by the second source when compared with electromagnetic radiation emitted by the first source, and wherein the second source emits electromagnetic radiation within one or more of: a near infrared waveband of the electromagnetic spectrum; or an infrared waveband of the electromagnetic spectrum.

Example 30 is a system as in any of Examples 21-29, wherein the pixel array is relatively inefficient at accumulating electromagnetic radiation emitted by the second source when compared with electromagnetic radiation emitted by the first source, and wherein the first source emits one or more of: white light; or a partition of electromagnetic radiation from a visible waveband of the electromagnetic spectrum.

Example 31 is a system as in any of Examples 21-30, wherein the electromagnetic radiation emitted by the second source comprises a shorter amplitude when compared with the electromagnetic radiation emitted by the first source.

Example 32 is a system as in any of Examples 21-31, wherein the electromagnetic radiation emitted by the second source comprises less energy when compared with the electromagnetic radiation emitted by the first source.

Example 33 is a system as in any of Examples 21-32, wherein the electromagnetic radiation emitted by the second source is dimmer than the electromagnetic radiation emitted by the first source.

Example 34 is a system as in any of Examples 21-33, wherein the first source and the second source emit different wavebands of the electromagnetic spectrum, and wherein the pixel array is inherently less efficient at accumulating a waveband of electromagnetic radiation emitted by the second source when compared with a waveband of electromagnetic radiation emitted by the first source.

Example 35 is a system as in any of Examples 21-34, wherein the image sensor reads out a color data frame and an advanced data frame, and wherein: the pixel array accumulates electromagnetic radiation resulting from an emission by the first source to output the color data frame; and the pixel array accumulates electromagnetic radiation resulting from an emission by the second source to output the advanced data frame.

Example 36 is a system as in any of Examples 21-35, wherein the advanced data frame comprises one or more of: a fluorescence data frame corresponding with the pixel array accumulating one or more of a fluorescence excitation wavelength of electromagnetic radiation or a fluorescence relaxation wavelength of electromagnetic radiation; or a multispectral data frame corresponding with the pixel array accumulating one or more of a multispectral wavelength of electromagnetic radiation or a spectral response emission of electromagnetic radiation.

Example 37 is a system as in any of Examples 21-36, wherein the pulse cycle is such that: the first source is cycled on during a blanking period immediately preceding readout of the color data frame; the first source is cycled off during a rolling readout sequence when the image sensor is reading out the color data frame; the first source is cycled off during a blanking period immediately preceding readout of the advanced data frame; and the first source is cycled off during a rolling readout sequence when the image sensor is reading out the advanced data frame.

Example 38 is a system as in any of Examples 21-37, wherein the pulse cycle is such that: the second source is cycled off during the blanking period immediately preceding the readout of the color data frame; the second source is cycled on during the rolling readout sequence when the image sensor is reading out the color data frame; the second source is cycled on during the blanking period immediately preceding the readout of the advanced data frame; and the second source is cycled on during the rolling readout sequence when the image sensor is reading out the advanced data frame.

Example 39 is a system as in any of Examples 21-38, wherein information from the color data frame and the advanced data frame are combined to generate an overlay frame, wherein the overlay frame comprises a color image depicting a scene and further comprises a false color overlay depicting information determined based on the advanced data frame.

Example 40 is a system as in any of Examples 21-39, wherein the color data frame further comprises advanced visualization data due to the emitter cycling on the second source during a readout period when the image sensor reads out the color data frame; and wherein the system further comprises a processor configured to subtract the advanced visualization data from the color data frame. Noise from the advanced data frame may be removed from the color frame with an algorithm configured to estimate the signal from each pixel of the advanced data frame that was collected during the readout period, and then subtracts this signal from the same pixel of the color data frame. For example, if a frame time consists of 60% blanking period and 40% readout period, then 40% of the signal collected by each pixel during the advanced data frame may be subtracted form the corresponding pixel of the color data frame.

It will be appreciated that various features disclosed herein provide significant advantages and advancements in the art. The following claims are exemplary of some of those features.

In the foregoing Detailed Description of the Disclosure, various features of the disclosure are grouped together in a single embodiment for the purpose of streamlining the disclosure. This method of disclosure is not to be interpreted as reflecting an intention that the claimed disclosure requires more features than are expressly recited in each claim. Rather, inventive aspects lie in less than all features of a single foregoing disclosed embodiment.

It is to be understood that any features of the above-described arrangements, examples, and embodiments may be combined in a single embodiment comprising a combination of features taken from any of the disclosed arrangements, examples, and embodiments.

It is to be understood that the above-described arrangements are only illustrative of the application of the principles of the disclosure. Numerous modifications and alternative arrangements may be devised by those skilled in the art without departing from the spirit and scope of the disclosure and the appended claims are intended to cover such modifications and arrangements.

Thus, while the disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

Further, where appropriate, functions described herein can be performed in one or more of: hardware, software, firmware, digital components, or analog components. For example, one or more application specific integrated circuits (ASICs) or field programmable gate arrays (FPGAs) can be programmed to carry out one or more of the systems and procedures described herein. Certain terms are used throughout the following description and claims to refer to particular system components. As one skilled in the art will appreciate, components may be referred to by different names. This document does not intend to distinguish between components that differ in name, but not function.

The foregoing description has been presented for the purposes of illustration and description. It is not intended to be exhaustive or to limit the disclosure to the precise form disclosed. Many modifications and variations are possible in light of the above teaching. Further, it should be noted that any or all the aforementioned alternate implementations may be used in any combination desired to form additional hybrid implementations of the disclosure.

Further, although specific implementations of the disclosure have been described and illustrated, the disclosure is not to be limited to the specific forms or arrangements of parts so described and illustrated. The scope of the disclosure is to be defined by the claims appended hereto, any future claims submitted here and in different applications, and their equivalents.

What is claimed is:

1. A system comprising:
an image sensor comprising a pixel array; and
an emitter comprising a plurality of sources of electromagnetic radiation, wherein the plurality of sources comprises a first source and a second source;
wherein the emitter cycles at least a portion of the plurality of sources according to a pulse cycle comprising:
a plurality of pulsed emissions by the first source; and
a plurality of pulsed emissions by the second source;
wherein at least a portion of the plurality of pulsed emissions by the second source overlap a readout period of the image sensor when the image sensor reads out a data frame corresponding with an emission by the first source; and
wherein the second source cycles off during at least a portion of the plurality of pulsed emissions by the first source.

2. The system of claim 1, wherein the first source is a visible source that pulses electromagnetic radiation within a visible waveband of the electromagnetic spectrum;
wherein the data frame corresponding with the emission by the first source is a color data frame;
wherein the pixel array accumulates reflected electromagnetic radiation during a blanking period when the emitter pulses the visible source; and
wherein the image sensor reads out the color data frame during a readout period immediately subsequent to the blanking period when the emitter pulses the visible source.

3. The system of claim 2, wherein the second source is an excitation source configured to emit a fluorescence excitation wavelength of electromagnetic radiation;
wherein the fluorescence excitation wavelength of electromagnetic radiation is selected to cause one or more of a reagent or a tissue to fluoresce;
wherein the pixel array accumulates a fluorescence relaxation wavelength of electromagnetic radiation emitted by the one or more of the reagent or the tissue; and
wherein the image sensor reads out a fluorescence data frame during a readout period corresponding with the second source.

4. The system of claim 3, wherein the second source is cycled on during each of:
the readout period when the image sensor reads out the color data frame; and
a blanking period immediately subsequent to the image sensor reading out the color data frame.

5. The system of claim 4, wherein the second source is further cycled on during the readout period corresponding with the second source wherein the image sensor reads out the fluorescence data frame; and
wherein the second source is cycled on continuously throughout each of:
the readout period when the image sensor reads out the color data frame;
the blanking period immediately subsequent to the image sensor reading out the color data frame; and
the readout period wherein the image sensor reads out the fluorescence data frame.

6. The system of claim 5, wherein the emitter cycles off the first source during each of:
the readout period when the image sensor reads out the color data frame;
the blanking period immediately subsequent to the image sensor reading out the color data frame; and
the readout period wherein the image sensor reads out the fluorescence data frame.

7. The system of claim 1, wherein the emitter cycles the first source and the second source on and off according to the pulse cycle such that the plurality of pulsed emissions by the first source do not overlap with the plurality of pulsed emissions by the second source.

8. The system of claim 1, further comprising:
an endoscope, wherein the image sensor is disposed within a distal region of the endoscope; and
a prism disposed within the distal region of the endoscope configured to reflect electromagnetic radiation on to the image sensor;
wherein a planar side of the pixel array is oriented parallel to a longitudinal axis of the endoscope.

9. The system of claim 1, wherein the pixel array is relatively inefficient at accumulating electromagnetic radiation emitted by the second source when compared with electromagnetic radiation emitted by the first source, and wherein the second source emits electromagnetic radiation within one or more of:
a near infrared waveband of the electromagnetic spectrum; or
an infrared waveband of the electromagnetic spectrum.

10. The system of claim 1, wherein the pixel array is relatively inefficient at accumulating electromagnetic radiation emitted by the second source when compared with electromagnetic radiation emitted by the first source, and wherein the first source emits one or more of:
white light; or
a partition of electromagnetic radiation from a visible waveband of the electromagnetic spectrum.

11. The system of claim 1, wherein the electromagnetic radiation emitted by the second source comprises a shorter amplitude when compared with the electromagnetic radiation emitted by the first source.

12. The system of claim 1, wherein the electromagnetic radiation emitted by the second source comprises less energy when compared with the electromagnetic radiation emitted by the first source.

13. The system of claim 1, wherein the electromagnetic radiation emitted by the second source is dimmer than the electromagnetic radiation emitted by the first source.

14. The system of claim 1, wherein the first source and the second source emit different wavebands of the electromagnetic spectrum, and wherein the pixel array is inherently less efficient at accumulating a waveband of electromagnetic radiation emitted by the second source when compared with a waveband of electromagnetic radiation emitted by the first source.

15. The system of claim 1, wherein the image sensor reads out a color data frame and an advanced data frame, and wherein:
the pixel array accumulates electromagnetic radiation resulting from an emission by the first source to output the color data frame; and
the pixel array accumulates electromagnetic radiation resulting from an emission by the second source to output the advanced data frame.

16. The system of claim 15, wherein the advanced data frame comprises one or more of:
a fluorescence data frame corresponding with the pixel array accumulating one or more of a fluorescence excitation wavelength of electromagnetic radiation or a fluorescence relaxation wavelength of electromagnetic radiation; or
a multispectral data frame corresponding with the pixel array accumulating one or more of a multispectral wavelength of electromagnetic radiation or a spectral response emission of electromagnetic radiation.

17. The system of claim 16, wherein the pulse cycle is such that:
the first source is cycled on during a blanking period immediately preceding readout of the color data frame;
the first source is cycled off during a rolling readout sequence when the image sensor is reading out the color data frame;
the first source is cycled off during a blanking period immediately preceding readout of the advanced data frame; and
the first source is cycled off during a rolling readout sequence when the image sensor is reading out the advanced data frame.

18. The system of claim 17, wherein the pulse cycle is such that:
the second source is cycled off during the blanking period immediately preceding the readout of the color data frame;
the second source is cycled on during the rolling readout sequence when the image sensor is reading out the color data frame;
the second source is cycled on during the blanking period immediately preceding the readout of the advanced data frame; and
the second source is cycled on during the rolling readout sequence when the image sensor is reading out the advanced data frame.

19. The system of claim 16, wherein information from the color data frame and the advanced data frame are combined to generate an overlay frame, wherein the overlay frame comprises a color image depicting a scene and further comprises a false color overlay depicting information determined based on the advanced data frame.

20. The system of claim 16, wherein the color data frame further comprises advanced visualization data due to the emitter cycling on the second source during a readout period when the image sensor reads out the color data frame; and
wherein the system further comprises a processor configured to subtract the advanced visualization data from the color data frame.
